(19) United States
(12) Patent Application Publication
(10) Pub. No.: US 2010/0189798 A1
(43) Pub. Date: Jul. 29, 2010

(54) CONTROL OF ERWINIA AMYLOVORA WITH VINYLGLYCINES AND BACTERIA THAT PRODUCE VINYLGLYCINES

(75) Inventors: Donald J. Armstrong, Corvallis, OR (US); Mark D. Azevedo, Albany, OR (US); Kerry L. McPhail, Corvallis, OR (US); Dallice I. Mills, Corvallis, OR (US); Gary Banowetz, Corvallis, OR (US); Anne B. Halgren, Corvallis, OR (US); Michael D. Jackson, Salem, OR (US); Muralidhara Thimmaiah, Corvallis, OR (US)

Correspondence Address: KLARQUIST SPARKMAN, LLP, 121 SW SALMON STREET, SUITE 1600, PORTLAND, OR, 97204, US

(73) Assignees: The State of Oregon acting by and through the State Board of Higher Education on behalf of; Oregon State University; The Government of the United States of America as represented by the Secretary of the Department of; Agriculture

(21) Appl. No.: 12/695,977

(22) Filed: Jan. 28, 2010

Related U.S. Application Data
(60) Provisional application No. 61/148,286, filed on Jan. 29, 2009.

(51) Int. Cl. A61K 9/16; A01N 37/44; A01N 63/00

(52) U.S. Cl. 424/490; 514/564; 424/93.47; 424/93.48

(57) ABSTRACT

This disclosure relates to methods of controlling Erwinia amylovora with natural and synthetically-produced vinylglycines and variants thereof. As Erwinia amylovora is the causal agent of the plant disease fire blight, by extension, this disclosure relates to methods of controlling fire blight with vinylglycines and variants thereof. This disclosure also relates to methods of controlling Erwinia amylovora, and by extension, fire blight, with vinylglycine-producing bacteria.

### CROSS REFERENCE TO RELATED APPLICATION

Benefit is claimed to U.S. Provisional Application No. 61/148,286, filed Jan. 29, 2009, which is incorporated herein by reference in its entirety.

### ACKNOWLEDGMENT OF GOVERNMENT SUPPORT

The claimed invention was developed, at least in part, with United States government support under grants received from the USDA-GSCSSA Grant Program (Grant Numbers G001225, G001340, G001579, G001706, G001850, G002053, and G002390). The United States government has certain rights in the invention.

### FIELD

This disclosure relates to methods of controlling Erwinia amylovora with natural and synthetically-produced vinylglycines and variants thereof. As Erwinia amylovora is the causal agent of fire blight, by extension, this disclosure relates to methods of controlling fire blight with vinylglycines and variants thereof. This disclosure also relates to methods of controlling Erwinia amylovora, and by extension, fire blight, with vinylglycine-producing bacteria.

### BACKGROUND

Erwinia amylovora, a Gram-negative bacterium in the family Enterobacteriaceae, is the causal agent in the disease of orchard crops known as fire blight (Eastgate, Mol. Plant Path., 1: 325-329, 2000). Fire blight is a destructive necrotic plant disease that affects a wide range of hosts in the rose family and is a significant problem in pear and apple orchards. Fire blight is distributed throughout the pear and apple growing regions of the world (at least 40 countries) and is particularly destructive in the warmer and wetter regions of Southern Europe and the Western and Midwestern United States. The disease severely limits commercial apple and pear production in these regions. For example, the 2000 fire blight epidemic in Southwest Michigan killed 250,000 apple trees causing $42 million in economic loss to the region. In less than two weeks this single epidemic event devastated the region's apple industry (on-line at canr.msu.edu/vanburen/fb2000.htm).

E. amylovora initially colonizes the surface of the stigma during early fruit bloom and is then washed to the base of the flower where infections occur. Symptoms of the disease include water soaking of the blossoms and leaf buds followed by rapid wilting and necrosis. The resulting scorched and blackened appearance inspired the name of fire blight. E. amylovora infections can rapidly spread throughout an orchard, killing flowers, developing fruit, leaves, shoots, branches and sometimes mature trees. Virtually all of the commercial apple and pear cultivars grown throughout the world are moderately to severely susceptible to infection by E. amylovora.

Traditional control of fire blight involved spray application of the antibiotic streptomycin. However, the occurrence of streptomycin-resistant E. amylovora populations, combined with the banning or strict regulation of its use, has made it imperative that alternative control approaches be found. While copper and the bacteriostatic antibiotic oxytetracycline (registered only for use on pear trees) are also used to control fire blight, they are considered less effective than streptomycin for suppression of antibiotic-sensitive E. amylovora populations.

Biological control measures for fire blight disease have been developed in response to the emergence of streptomycin-resistant strains of E. amylovora. Such biological control measures involve the use of organisms that provide protection against blossom infections by competing with the pathogen for space and/or resources on the stigma of the blossoms, or that produce metabolites (other than vinylglycines) that are toxic to E. amylovora. Pseudomonas fluorescens A506 (“BlightBan® A506”, Nufarm®, USA) and Bacillus subtilis QST 713 (“Serenade®”, Agraquest®, USA) are currently registered in the United States for use as biocontrol agents for fire blight. BlightBan® A506 is described as an antagonistic biocontrol agent, while Serenade® controls E. amylovora by producing antimicrobial lipopeptide metabolites.

Successful biocontrol measures require that the beneficial microorganism colonize the flowers' stigmatic surfaces at levels sufficient to exclude or inhibit E. amylovora. The success of this inundative biocontrol approach is dependent upon several interactive and complex variables. Weather conditions throughout the bloom period, timing of application of the biocontrol agents, host susceptibility to fire blight, and the relative fitness of the biocontrol agents all impact the success of these treatments. Because of these and other factors, the use of available biocontrol agents in fire blight management has had limited success. Treatment typically reduces the disease severity by no more than 40-60%. This may not be an acceptable level of control, particularly during years of heavy disease pressure. There is therefore a demonstrated need to develop other methods of controlling E. amylovora, and by extension, fire blight.

### SUMMARY

Described herein are methods of inhibiting the growth of Erwinia amylovora, and by extension of controlling fire blight disease, on a plant. The methods inhibit E. amylovora growth through application of one or more vinylglycines, one or more vinylglycine variants (e.g., structural or mechanistic variants), or bacteria that produce such molecules. As described herein, such application may be in the form of any suitable formulation or agronomically-compatible composition.

Fire blight afflicts plants of the rose family. Thus the disclosed methods are applicable to controlling fire blight on members of the rose family, including but not limited to apple and pear trees, crabapples, quinces, loquats, cotoneaster and pyracantha, roses, raspberries, blackberries, and so forth.

One embodiment is a method of inhibiting growth of E. amylovora on a plant comprising applying to the plant an effective amount of a natural or synthetic formylaminooxyvinylglycine (FVG) or a structurally related analog thereof. The disclosure also provides methods of inhibiting E. amylovora growth on a plant by application of a bacterial strain that is producing FVG or a structural analog of FVG. Also disclosed herein is the use of particular isolates of FVG-producing strains of Pseudomonas fluorescens, Pseudomonas mucidolens/synxantha, and Enterobacter kobei as biocontrol agents of E. amylovora growth.

Also provided are methods of inhibiting growth of E. amylovora on a plant by applying to the plant an effective amount of a non-vinylglycine inhibitor of pyridoxal-phosphate-dependent enzyme reactions.

A further embodiment of the method of inhibiting growth of E. amylovora on a plant comprises applying to the plant an effective amount of aminoethoxyvinylglycine (AVG), or a structurally related compound, prior to fruit set and petal fall from the plant. Similarly, any bacterial strain that is producing AVG or a structurally related compound can be applied to a plant prior to fruit set and petal fall to inhibit the growth of E. amylovora.

The foregoing and other objects, features, and advantages will become more apparent from the following detailed description, which proceeds with reference to the accompanying figures.

### BRIEF DESCRIPTION OF THE FIGURES

FIG. 1 shows a comparison of the effects on Erwinia amylovora of culture filtrates from Pseudomonas fluorescens WH6 and the GAF2 mutant of WH6. Petri plates were prepared and inoculated with E. amylovora as described in Example 1. The central well in each freshly inoculated plate received 300 μL of the indicated culture filtrate, and the cultures were incubated at 28° C. for 48 hours prior to evaluation.

FIG. 2 shows a comparison of the effects on Erwinia amylovora of culture filtrates from Pseudomonas fluorescens WH6 and the GAF3 mutant of WH6. Petri plates were prepared and inoculated with E. amylovora as described in Example 1. The central well in each freshly inoculated plate received 300 μL of the indicated culture filtrate, and the cultures were incubated at 28° C. for 48 hours prior to evaluation.

FIG. 3 shows a comparison of the effects on Erwinia amylovora of live bacterial colonies of Pseudomonas fluorescens WH6 and the GAF2 mutant of WH6. Petri plates containing 925 Minimal Culture Medium were prepared as described in Example 1. Samples of live Pseudomonas bacteria were inoculated at the sites indicated immediately after each plate was spread with a lawn of E. amylovora inoculum. Inocula were prepared and inoculations performed as described in Example 1. The cultures were incubated at 28° C. for 48 hours prior to evaluation.

FIG. 4 shows the effects on Erwinia amylovora of culture filtrates from the FVG-producing Pseudomonas isolates described in U.S. Patent Publication 2006/0147438. Petri plates were prepared and inoculated with E. amylovora as described in Example 1. The central well or wells in each freshly inoculated plate received 300 μL of the indicated culture filtrate, and the cultures were incubated at 28° C. for 48 hours prior to evaluation.

FIG. 5 shows the effects on Erwinia amylovora of live bacterial colonies from the FVG-producing Pseudomonas isolates described in US Patent Publication 2006/0147438. Petri plates containing 925 Minimal Culture Medium were prepared as described in Example 1. Samples of live Pseudomonas bacteria were inoculated at the sites indicated immediately after each plate was spread with a lawn of E. amylovora inoculum. Inocula were prepared and inoculations performed as described in Example 1. The cultures were incubated at 28° C. for 48 hours prior to evaluation.

FIG. 6 shows the effects on Erwinia amylovora of live bacterial colonies and culture filtrates from several Pseudomonas fluorescens isolates. Petri plates containing 925 Minimal Culture Medium were prepared as described in Example 1. Samples of live Pseudomonas bacteria were inoculated (right side) immediately after each plate was spread with a lawn of E. amylovora inoculum. Inocula were prepared and inoculations performed as described in Example 1. The well in each freshly inoculated plate received 300 μL of the indicated culture filtrate (left side). The cultures were incubated at 28° C. for 48 hours prior to evaluation.

FIG. 7 shows the effects on Erwinia amylovora of live bacterial colonies and culture filtrates from Pseudomonas laboratory strains. Petri plates containing 925 Minimal Culture Medium were prepared as described in Example 1. Samples of live Pseudomonas bacteria were inoculated at the sites indicated immediately after each plate was spread with a lawn of E. amylovora inoculum. Inocula were prepared and inoculations performed as described in Example 1. The well in each freshly inoculated plate received 300 μL of the indicated culture filtrate. The cultures were incubated at 28° C. for 48 hours prior to evaluation.

FIG. 8 shows a Thin-Layer Chromatography (TLC) analysis of culture filtrates from several Pseudomonas fluorescens isolates. Culture filtrates were prepared, extracted, and chromatographed on cellulose TLC plates as described in Example 1. The developed chromatograms were sprayed with ninhydrin reagent as described in the same source. The chromatogram prepared from WH6 culture filtrate is included as a reference. The ninhydrin-positive band labeled as GAF (Germination-Arrest Factor) has been identified as FVG (formylaminooxyvinylglycine) as described in U.S. Patent application Ser. No. 12/567,590. FIG. 8A shows chromatograms from strains WH6, AH10, BT1, and E24. FIG. 8B shows chromatograms from strains TDH5, TR33, TR44, and TR46.

FIG. 9 shows a Thin-Layer Chromatography (TLC) analysis of culture filtrates from Pseudomonas strains obtained from other laboratories. Culture filtrates were prepared, extracted, and chromatographed on cellulose TLC plates as described in Example 1. The developed chromatograms were sprayed with ninhydrin reagent as described in the same source. The chromatogram prepared from WH6 culture filtrate is included as a reference. The ninhydrin-positive band labeled as GAF (Germination-Arrest Factor) has been identified as FVG (formylaminooxyvinylglycine) as described in U.S. patent application Ser. No. 12/567,590.

FIG. 10 shows the ¹H NMR spectrum (in D₂O, at 300 MHz) for purified GAF (FVG). Arrows indicate characteristic FVG peaks.

FIG. 11 shows the ¹H NMR spectrum (in D₂O, at 300 MHz) for a crude 90% ethanol extract of the culture filtrate from Pseudomonas fluorescens Isolate WH6. Arrows indicate characteristic FVG peaks.

FIG. 12 shows the ¹H NMR spectrum (in D₂O, at 300 MHz) for a crude 90% ethanol extract of the culture filtrate from Pseudomonas fluorescens Isolate GTR24. Arrows indicate characteristic FVG peaks.

FIG. 13 shows the ¹H NMR spectrum (in D₂O, at 300 MHz) for a crude 90% ethanol extract of the culture filtrate from Enterobacter kobei Isolate A3203. Arrows indicate characteristic FVG peaks.

FIG. 14 shows the ¹H NMR spectrum (in D₂O, at 300 MHz) for a crude 90% ethanol extract of the culture filtrate from Pseudomonas fluorescens Isolate TDH5.

FIG. 15 shows the ¹H NMR spectrum (in D₂O, at 300 MHz) for a crude 90% ethanol extract of the culture filtrate from Pseudomonas fluorescens Strain PfO-1.

FIG. 16 shows the effect of ReTain® at a concentration equivalent to 30 mM aminoethoxyvinylglycine (AVG) on Erwinia amylovora. Petri plates were prepared and inoculated with E. amylovora as described in Example 1. The central well in each freshly inoculated plate received 300 μL of 30 mM AVG (as ReTain®). The cultures were then incubated for 28° C. for 48 hours prior to evaluation.

FIG. 17 shows the effect of varying ReTain® concentrations equivalent to 0.1 to 3 mM aminoethoxyvinylglycine (AVG) on Erwinia amylovora. Petri plates were prepared and inoculated with E. amylovora as described in Example 1. The central well in each freshly inoculated plate received 300 μL of the indicated concentration of AVG (as ReTain®). The cultures were then incubated at 28° C. for 48 hours prior to evaluation.

FIG. 18 shows the effect of a sample of pure aminoethoxyvinylglycine-HCl (AVG-HCl) on Erwinia amylovora. Petri plates were prepared and inoculated with E. amylovora as described in Example 1. The central well in each freshly inoculated plate received 300 μL of 3 mM AVG-HCl, supplied as a pure commercial sample of the compound. The cultures were then incubated for 28° C. for 48 hours prior to evaluation.

FIG. 19 shows a comparison of the effects of aminooxyacetic acid (AOA) and aminoethoxyvinylglycine (AVG, as Retail®) on Erwinia amylovora. Petri plates were prepared and inoculated with E. amylovora as described in Example 1. The central well in each freshly inoculated plate received 300 μL of the indicated concentration of AOA or AVG (as ReTain®). The cultures were then incubated at 28° C. for 48 hours prior to evaluation.

### DETAILED DESCRIPTION

### I. Abbreviations

AOA: aminooxyacetic acid, 2-aminooxyacetic acid
AVG: aminoethoxyvinylglycine, 2-amino-4-aminoethoxybut-3-enoic acid
FVG: formylaminooxyvinylglycine, 2-amino-4-formylaminooxybut-3-enoic acid
FPM: fluorescent Pseudomonas Medium

### GAF: Germination-Arrest Factor

NMR: Nuclear magnetic resonance

### PMS: Pseudomonas Minimal Salt Medium

SCFU: single colony forming units
TLC: thin-layer chromatography

UV: ultraviolet

### II. Terms

Unless otherwise noted, technical terms are used according to conventional usage. In order to facilitate review of the various embodiments of this disclosure, the following explanations of specific terms are provided:

Adjuvant: A chemical added to an antimicrobial agent, herbicide, or pesticide formulation or tank mix to improve mixing and application or enhance performance. Most formulations contain at least a small percentage of adjuvants. Wetting agents and spreaders are the adjuvants most frequently added. Common adjuvants include, but are not limited to, wetting agents, such as anionic, cationic, nonionic, and amphoteric surfactants, stabilizers, preservatives, antioxidants, extenders, solvents, emulsifiers, invert emulsifiers, spreaders, stickers, penetrants, foaming agents, anti-foaming agents, thickeners, safeners, compatibility agents, crop oil concentrates, viscosity regulators, binders, tackers, drift control agents, or other chemical agents, such as fertilizers, antibiotics, fungicides, nematicides, or pesticides.

Antibiotic: A substance, for example penicillin or streptomycin, often produced by or derived from certain fungi, bacteria, and other organisms, that can destroy or inhibit the growth of other microorganisms.

Anti-foaming agent: An adjuvant that reduces foaming of spray mixtures that require vigorous agitation.

Antioxidant: An adjuvant, such as vitamin E, vitamin C, or beta carotene that protects a compound, for example an antimicrobial agent, herbicide, or pesticide from the damaging effects of oxidation.

Biocontrol agent: A preparation of living inoculum of a microorganism that is formulated and applied in a manner analogous to that of a chemical agent in an effort to suppress the growth of plant pathogens or other undesirable organisms. Generally, the application of a biocontrol agent is timed to take advantage of favorable environmental conditions and/or the most susceptible stage of pathogen infection. Similarly, the biocontrol agent generally is formulated to avoid unfavorable environmental conditions and to facilitate application.

Buffer: An ionic compound that resists changes in its pH.

Compatibility Agent: An adjuvant that aids in combining antimicrobial agents, herbicides, or pesticides effectively.

Crop Oil Concentrate: A phytobland petroleum or vegetable oil that increases absorption of an antimicrobial agent, herbicide, or pesticide, and aids in moving these substances across the target surfaces as well as in reducing surface tension of spray droplets. Crop oils are effective in increasing spray retention on target surfaces and in reducing drying times. Crop oil concentrates may also contain up to 20% surfactant.

Drift Control Agent: An adjuvant used to reduce the fine particles in the spray pattern that are responsible for drift of antimicrobial agents, herbicides, or pesticides onto nontreated areas with potential deleterious results.

Dry flowable: A granule formulation similar to a wettable powder, except that the active ingredient is formulated on a large particle (granule) instead of onto a ground powder. This type of formulation offers essentially the same advantages and disadvantages as wettable powder formulations. However, these formulations generally are more easily mixed and measured than wettable powders. Because they create less dust when handling, they cause less inhalation hazard to the applicator during pouring and mixing.

Effective amount: An “effective amount” of a compound refers to an amount of a compound that will inhibit the growth of at least some of the Erwinia amylovora in a sample or on a plant that is exposed to the particular compound. For example, the effective amount of FVG may be an amount sufficient to inhibit the growth of some of the E. amylovora that is present on a plant. In specific embodiments of the disclosure, an effective amount of FVG inhibits the growth of at least 10% of the Erwinia in a given sample or on a given plant. In particular embodiments of the disclosure, an effective amount of FVG inhibits growth in at least 20%, or even 50%, of the Erwinia in a given sample or on a given plant. In more particular embodiments of the disclosure, an effective amount of FVG inhibits growth of over 90%, or nearly 100% of the Erwinia in a given sample. Specific examples of effective amounts of FVG are provided in the Examples below.

Emulsifiable concentrate: A liquid formulation of an antimicrobial agent, herbicide, or pesticide that contains the active ingredient, one or more solvents, and an emulsifier that allows mixing with water. Formulations of this type are highly concentrated, relatively inexpensive per pound of active ingredient, and easy to handle, transport, and store. In addition, they require little agitation (will not settle out or separate) and are not abrasive to machinery or spraying equipment.

Emulsifier: A substance that promotes the suspension of one liquid in another. Emulsifiers are often used to disperse petroleum-based antimicrobial agents, herbicides, or pesticides in water.

Erwinia amylovora: A Gram-negative bacterium in the family Enterobacteriaceae that is the causal agent of the plant disease fire blight.

Extender: An adjuvant added to an antimicrobial agent, herbicide, or pesticide to modify, dilute, or adulterate it.

Fire blight: A destructive necrotic plant disease, caused by Erwinia amylovora, which affects a wide range of hosts in the rose family and is a significant problem in pear and apple orchards. Fire blight is distributed throughout the pear and apple growing regions of the world (at least 40 countries) and is particularly destructive in the warmer and wetter regions of Southern Europe and the Western and Midwestern United States.

Foaming agent: An adjuvant used to reduce foaming in a spray system so that pumps and nozzles can operate properly.

Fungicide: A chemical substance that destroys or inhibits the growth of fungi.

Germination-Arrest Factor: A naturally occurring herbicide produced by certain isolates of soil bacteria and identified as FVG (formylaminooxyvinylglycine) as described in U.S. patent application Ser. No. 12/567,590.

Granule: A formulation of an antimicrobial agent, herbicide, or pesticide in which the active ingredient is formulated onto large particles (granules). The primary advantages of this type of formulation are that the formulation is ready to use with simple application equipment (seeders or spreaders), and the drift potential is low because the particles are large and settle quickly. The disadvantage of this formulation is that it does not usually adhere to foliage.

Harvest: A time period associated with the harvesting of agricultural products. The time of harvest for any given crop is specific to the crop, climate, and farming context and is determined by the desired condition of the product to be harvested and the distance between the farm and its intended market.

Inhibit: Slow or stop the growth of an organism. In particular examples, the growth of Erwinia amylovora is inhibited by contact with an antimicrobial agent such as FVG, a FVG derivative, or a FVG-producing bacterium.

Invert emulsifier: An adjuvant that allows water-based antimicrobial agents, herbicides, or pesticides to mix with a petroleum carrier.

Isolated: An isolated biological agent or component has been substantially separated, produced apart from, or purified away from other biological agents or components in the environment or from a cell of the organism in which the agent or component naturally occurs. In some examples, an isolated biological agent or component is a nucleic acid, protein or modified amino acid that has been isolated and purified by standard purification methods. In other examples, it is an isolated bacterium that has been identified and cultured to homogeneity or near homogeneity by standard microbiological techniques. The term also embraces biological agents or components prepared by recombinant expression in a host cell as well as those that have been chemically synthesized extrabiologically. The term isolated does not require absolute purity; rather, this is intended as a relative term. Thus, for example, an isolated vinylglycine is one in which the vinylglycine is more enriched than in its natural environment within a cell. Preferably, an isolated preparation is purified such that the vinylglycine is at least 50% of the total biochemical content of the preparation.

Liquid flowable: A formulation of an antimicrobial agent, herbicide, or pesticide that is made up of finely ground active ingredient suspended in a liquid. Flowables generally are mixed with water for application, are easily handled and applied, and seldom clog nozzles. Some of their disadvantages are that they may leave a visible residue on plant and soil surfaces, and typically require constant and thorough agitation to remain in suspension.

Nematicide: A substance or preparation used to kill nematodes.

Ninhydrin: A chemical compound with the molecular formula C₉H₆O₄, ninhydrin is also known as ninhydrin monohydrate, 1,2,3-triketohydrindene monohydrate, 1,2,3-indantrione monohydrate, 2,2-dihydroxy-1,3-indandione, 1H-indene-1,2,3-trione monohydrate. Ninhydrin produces a purple reaction product in the presence of primary amines. A ninhydrin-positive or ninhydrin-reactive agent is one that produces such a reaction product, and the presence of such a reaction product indicates that the agent includes at least one primary amine. Thus, a ninhydrin-reactive agent is one that includes at least one primary amine, for example an amino acid, peptide, protein, or another agent that includes at least one primary amine, such as an enzymatically synthesized or modified agent.

Pellet: A formulation of an antimicrobial agent, herbicide, or pesticide in which the active ingredient is formulated onto large particles (pellets). The primary advantages of this type of formulation are that the formulation is ready to use with simple application equipment (seeders or spreaders), and the drift potential is low because the particles are large and settle quickly. The disadvantage of this formulation is that it does not adhere to foliage.

Penetrant: An adjuvant that allows an antimicrobial agent, herbicide, or pesticide to get through the outer surface to the inside of a treated area.

Pesta: A granular product made from cereal grain flour and a biocontrol agent. The process encapsulates biocontrol agents in pasta-like products called pesta (U.S. Pat. No. 5,074,902; and Connick et al., J. Nematol., 25(2):198-203, 1993. Bacteria formulated in such media may exhibit extended shelf and field-life (e.g., Connick et al., Am. Biotechnol. Lab. 14:34-37, 1996). These characteristics are desired in a product which may be stored prior to use or shipped over long-distances prior to being used in the field. Therefore, the biocontrol compositions of the present invention may be formulated in a suitable composition, for example, but not limited to, pesta.

Petal fall: A designation for a particular point of time in the growing season of a blooming plant. Petal fall occurs following pollination and prior to fruit set, and is marked by the loss of petals from the plant flower. In orchard plants, for example apple and pear trees, petal fall occurs before maturation of developing fruit. In all instances, petal fall occurs more than six weeks before harvest.

Preservative: An adjuvant that inhibits degradation of an antimicrobial agent, herbicide, or pesticide.

Purified: The term “purified” does not require absolute purity; rather, it is intended as a relative term. Thus, for example, a purified protein preparation is one in which the protein referred to is more pure than the protein in its natural environment within a cell.

Safener: An adjuvant that reduces the toxicity of a formulation of an antimicrobial agent, herbicide, or pesticide to the handler or to the crop.

Soluble powder: An antimicrobial, herbicide, or pesticide formulation that, when mixed with water, dissolves readily and forms a true solution. Soluble powder formulations are dry and include the active ingredient and additives. When thoroughly mixed, no further agitation is necessary to keep the active ingredient dissolved in solution.

Solution: A liquid formulation that includes an active ingredient and an additive. Solution formulations are designed for those active ingredients that dissolve readily in water. Generally, when active ingredients formulated as solutions are mixed with water, the active ingredient will not settle out of solution or separate.

Solvent: A substance (usually liquid) suitable for, or employed in solution, or in dissolving something. For example, water is an appropriate solvent of most salts; alcohol of resins; ether of fats; and mercury or acids of metals.

Spreader: An adjuvant that allows an antimicrobial agent, herbicide, or pesticide to form a uniform coating layer over the treated surface.

Stabilizer: A substance that renders or maintains a solution, mixture, suspension, or state resistant to chemical change.

Sticker: An adjuvant that causes an antimicrobial agent, herbicide, or pesticide to adhere to plant foliage. Stickers reduce spray runoff during application and washoff by rain. Many stickers are blended with wetting agents so that they both increase spray coverage and provide better adhesion. These combined products are often called “spreader-stickers”.

Surfactant: A type of adjuvant designed to improve the dispersing/emulsifying, absorbing, spreading, sticking and/or pest-penetrating properties of an antimicrobial agent, herbicide, or pesticide formulation. Surfactants can be divided into the following five groupings: 1) non-ionic surfactants, 2) crop oil concentrates, 3) nitrogen-surfactant blends, 4) esterified seed oils, and 5) organo-silicones.

Suitable surfactants may be nonionic, cationic, or anionic, depending on the nature of the compound used as an active ingredient. Surfactants may be mixed together in some embodiments of the disclosure. Nonionic surfactants include polyglycol ether derivatives of aliphatic or cycloaliphatic alcohols, saturated or unsaturated fatty acids and alkylphenols. Fatty acid esters of polyoxyethylene sorbitan, such as polyoxyethylene sorbitan trioleate, also are suitable nonionic surfactants. Other suitable nonionic surfactants include water-soluble polyadducts of polyethylene oxide with polypropylene glycol, ethylenediaminopolypropylene glycol and alkylpolypropylene glycol. Particular nonionic surfactants include nonylphenol polyethoxyethanols, polyethoxylated castor oil, polyadducts of polypropylene and polyethylene oxide, tributylphenol polyethoxylate, polyethylene glycol and octylphenol polyethoxylate.

Cationic surfactants include quaternary ammonium salts carrying, as N-substituents, an 8 to 22 carbon straight or branched chain alkyl radical. The quaternary ammonium salts may carry additional N-substituents, such as unsubstituted or halogenated lower alkyl, benzyl, or hydroxy-lower alkyl radicals. Some such salts exist in the form of halides, methyl sulfates, and ethyl sulfates. Particular salts include stearyldimethylammonium chloride and benzyl bis(2-chloroethyl)ethylammonium bromide.

Suitable anionic surfactants may be water-soluble soaps as well as water-soluble synthetic surface-active compounds. Suitable soaps include alkali metal salts, alkaline earth metal salts, and unsubstituted or substituted ammonium salts of higher fatty acids. Particular soaps include the sodium or potassium salts of oleic or stearic acid, or of natural fatty acid mixtures. Synthetic anionic surfactants include fatty sulfonates, fatty sulfates, sulfonated benzimidazole derivatives, and alkylarylsulfonates. Particular synthetic anionic surfactants include the sodium or calcium salt of ligninsulfonic acid, of dodecyl sulfate, or of a mixture of fatty alcohol sulfates obtained from natural fatty acids. Additional examples include alkylarylsulfonates, such as sodium or calcium salts of dodecylbenzenesulfonic acid, or dibutylnaphthalenesulfonic acid. Corresponding phosphates for such anionic surfactants are also suitable.

Thickener: An adjuvant that reduces drift by increasing droplet size and reducing volume of spray contained in drift-prone droplets.

Timed-Release Coating: A coating on a solid or particulate anti-Erwinia formulation that retards degradation and prolongs anti-Erwinia activity of the antimicrobial component of the formulation. Coatings can be divided into three categories: (1) coatings that directly degrade in the presence of water, (2) coatings that are broken apart by wet and dry cycles, and (3) coatings degraded by specific temperatures, for example Degree Herbicide® by Monsanto.

Water-Resistant Coating: A coating on a solid or particulate anti-Erwinia formulation that repels water and delays dissolution of the antimicrobial formulation. One common technique used commercially is interfacial polycondensation of multifunctional isocyanates with multifunctional amines. In this technique, the oil phase containing the active agent and the isocyanate is emulsified in the aqueous phase containing the amine monomer. The isocyanate reacts with the amine at the oil-water interface to form a solid polyurea shell wall about the encapsulated active agent. A second technique involves coating an anti-Erwinia formulation, for example an FVG formulation, with whey protein that has been treated to provide a specific form or structure that is more highly resistant to dissolution in water.

Wettable powder: A dry, finely ground formulation of an antimicrobial agent, herbicide, or pesticide in which the active ingredient is combined with a finely ground carrier (usually mineral clay), along with other ingredients to enhance the ability of the powder to suspend in water. Generally, the powder is mixed with water for application. Wettable powders are one of the most widely used herbicide formulations and offer low cost and ease of storage, transport, and handling; lower phytotoxicity potential than emulsifiable concentrates and other liquid formulations; and less skin and eye absorption hazard than emulsifiable concentrates and other liquid formulations. Some disadvantages are that they require constant and thorough agitation in the spray tank, are abrasive to pumps and nozzles (causing premature wear), may produce visible residues on plant and soil surfaces, and can create an inhalation hazard to the applicator while handling (pouring and mixing) the concentrated powder. Typical solid diluents are described in Watkins et al., Handbook of Insecticide Dust Diluents and Carriers, 2nd Ed., Dorland Books, Caldwell, N.J. The more absorptive diluents are preferred for wettable powders and the denser ones for dusts.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The term “comprises” means “includes.” In case of conflict, the present specification, including explanations of terms, will control. In addition, the materials, methods, and examples are illustrative only and not intended to be limiting. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

### III. Overview of Several Embodiments

Provided herein in a first embodiment is a method of inhibiting growth of E. amylovora on a plant, comprising applying to the plant an effective amount of a natural or synthetic FVG or a structurally related analog thereof. In particular examples of that method, inhibiting growth of E. amylovora on a plant comprises applying to the plant a bacterial strain that is producing FVG or a structurally related analog thereof.

Provided in another embodiment is a method of inhibiting growth of E. amylovora on a plant, comprising applying to the plant an effective amount of a non-vinylglycine inhibitor of pyridoxal-phosphate-dependent enzyme reactions.

Yet another embodiment is a method of inhibiting growth of E. amylovora on a plant comprising applying to the plant an effective amount of AVG, or a structurally related compound, prior to fruit set and petal fall from the plant. Alternatively, the method of inhibiting growth of E. amylovora on a plant comprises applying to the plant a bacterial strain that is producing AVG, or a structurally related compound, prior to fruit set and petal fall from the plant.

In examples of the described methods, the plant has fire blight.

In examples of the described methods, the plant is an apple tree, a pear tree, or a member of family Rosaceae.

In some embodiments of the methods of inhibiting E. amylovora growth with a bacterial strain that is producing FVG or a structurally related analog thereof, the bacterial strain is an FVG-producing strain of Pseudomonas fluorescens, an FVG-producing strain of Pseudomonas mucidolens/synxantha, or an FVG producing strain of an Enterobacter kobei.

By way of example, the bacterial strain used in some of the described methods is selected from the group consisting of Pseudomonas fluorescens strain isolates WH6 (NRRL# B-30485), AD31 (NRRL# B-30483), AH4 (NRRL#B-30482), E34 (NRRL# B-30481), WH19 (NRRL# B-30484), AH10 (NRRL# B-50232), BT1 (NRRL#B-50230), E24 (NRRL# B-50229), TR33 (NRRL# B-50220), TR44 (NRRL# B-50219), TR46 (NRRL# B-50218), A3422A (NRRL# B-50234), ALW38 (NRRL# B-50231), G2Y (NRRL# B-50228), GTR12 (NRRL# B-50227), GTR24 (NRRL# B-50226), GTR40 (NRRL# B-50225), HB14 (NRRL# B-50224), HB26 (NRRL# B-50223), HB32 (NRRL# B-50222), ST22 (NRRL# B-50221), or W36 (NRRL# B-50217); or Pseudomonas mucidolens/synxantha A342 (NRRL# B-50236) or TDH40 (NRRL# B-50235); or Enterobacter kobei A3203 (NRRL# B-50233). Alternatively, mixtures of two or more thereof may be used.

Optionally, the bacteria are applied in an agronomically-compatible composition.

In a particular embodiment of the disclosed methods, the FVG, non-vinylglycine inhibitor of pyridoxal-phosphate-dependent enzyme reactions, AVG, or structurally related analog of FVG or AVG is applied in a formulation that also comprises a surfactant, a stabilizer, a buffer, a preservative, an antioxidant, an extender, a solvent, an emulsifier, an invert emulsifier, a spreader, a sticker, a penetrant, a foaming agent, an anti-foaming agent, a thickener, a safener, a compatibility agent, a crop oil concentrate, a viscosity regulator, a binder, a tacker, a drift control agent, a fertilizer, an antibiotic, a fungicide, a nematicide, or a pesticide.

In an example embodiment of the disclosed methods, the FVG, non-vinylglycine inhibitor of pyridoxal-phosphate-dependent enzyme reactions, AVG, or structurally related analog of FVG or AVG is applied in a formulation that is a solution, a soluble powder, an emulsifiable concentrate, a wettable powder, a liquid flowable, a dry flowable, a water-dispersible granule, a granule, or a pellet.

In another example embodiment, the FVG, non-vinylglycine inhibitor of pyridoxal-phosphate-dependent enzyme reactions, AVG, or structurally related analog of FVG or AVG is formulated as a granule. Such granule, in some embodiments, is at least partially coated with a timed- or temperature-release coating. Optionally, the timed- or temperature-release coating is itself coated with a water-resistant coating.

### IV. Methods of Controlling Fire Blight

Erwinia amylovora is a Gram-negative bacterium that is the causal agent of the plant disease fire blight. Fire blight afflicts members of the plant family Rosaceae, which includes agriculturally significant apple and pear trees. Fire blight is highly contagious, being spread through insects or rainfall. Although Erwinia is capable of colonizing any part of the plant, fire blight-causing infections most commonly begin on the plant flower, with the bacteria entering the rest of the plant through the nectary (Eastgate, Mol. Plant Path., 1: 325-329, 2000). This infection of the plant's flowers by Erwinia is referred to as the blossom blight phase of fire blight disease. Current methods of blossom blight prevention involve the use, individually or in combination, of antibiotics and competitive, non-pathogenic bacteria to inhibit Erwinia growth, but the increasing prevalence of antibiotic resistant strains of Erwinia have reduced the efficacy of such treatments.

Demonstrated herein is a novel method of controlling fire blight, based on the ability of vinylglycines, and in a particular embodiment FVG (formylaminooxyvinylglycine), to inhibit the growth of the causal agent of fire blight, E. amylovora. This ability of FVG was discovered in tests with culture filtrate from the FVG-producing bacterial isolate P. fluorescens WH6, and further demonstrated by direct inhibition of E. amylovora growth by live P. fluorescens WH6. The ability of FVG and FVG-producing strains of Pseudomonas to inhibit multiplication of E. amylovora suggested that other vinylglycines and vinylglycine-producing bacteria would be effective in inhibiting E. amylovora and by extension effective in controlling fire blight. As disclosed herein, this prediction was confirmed by demonstration that the known vinylglycine AVG (aminoethoxyvinylglycine) reproduced the unknown characteristic of inhibiting Erwinia.

FVG and other vinylglycines used as direct chemical agents for the control of fire blight have the potential of providing a viable alternative to the use of streptomycin. Moreover, in the field of plant disease management, there is increasingly an expectation for reliable control strategies that are both effective and environmentally safe. As disclosed herein, FVG and the other vinylglycines that have been tested appear to target and arrest the growth of a narrow group of microorganisms. As natural products with a specific mode of action, these compounds are also likely to have minimal potential environmental effects compared to the use of streptomycin.

Moreover, as disclosed herein, vinylglycine-producing microorganisms themselves may be alternatives to the current biocontrol agents used to control fire blight. Vinylglycine-producing organisms would be expected to target E. amylovora relatively specifically and to act via a biochemical mechanism that would be distinct from the mechanisms of action of any of the current biocontrol agents. Because of this unique mode of action, vinylglycine-producing organisms, whether used by themselves or in combination with other biocontrol agents, may provide enhanced efficacy in the control of fire blight disease.

### V. Methods of Inhibiting Erwinia amylovora Growth

Disclosed herein is a novel method of inhibiting Erwinia amylovora growth, and by extension, a method to control the plant disease fire blight. This method is based on the discovery that vinylglycines and vinylglycine producing bacteria are able to inhibit the growth of E. amylovora.

In a general embodiment, the growth of Erwinia amylovora is inhibited through application of an effective amount of the vinylglycine FVG (formylaminooxyvinylglycine) to E. amylovora. In another general embodiment of the invention, the growth of E. amylovora is inhibited through direct contact with a FVG-producing strain of bacteria. In both embodiments, when the E. amylovora is located on a blooming plant, the inhibition of E. amylovora with an effective amount of FVG or a FVG-producing bacteria results in the inhibition of fire blight disease in the plant and further, in the field or orchard in which the plant is located.

FVG is an inhibitor of pyridoxal-phosphate dependent enzyme reactions. Thus, in other embodiments, the inhibition of E. amylovora is achieved through application of an effective amount of a FVG structural analog such as AVG (aminoethoxyvinylglycine), or a FVG mechanistic analog such as aminooxyacetic acid (AOA) that similarly inhibits pyridoxal-phosphate-dependent enzyme reactions.

### A. FVG

In one embodiment of the disclosed method, the inhibition of E. amylovora growth is achieved through application of the naturally produced vinylglycine FVG (formylaminooxyvinylglycine). The inhibition of E. amylovora by FVG was discovered in experiments undertaken to characterize the physical and biological properties of Pseudomonas fluorescens strain WH6 culture filtrate. As disclosed in Example 1, culture filtrate from WH6 was found to inhibit growth of E. amylovora while exerting little effect on the growth of a number of other bacterial species screened for possible FVG sensitivity. Similar results were obtained with the live WH6 organism. The source of this antimicrobial activity was identified through studies involving WH6 mutants (GAF2 and GAF3, described in U.S. Patent Publication 2006/0147438, incorporated herein by reference in its entirety) that have lost the ability to produce FVG, and which were both unable to inhibit the growth of E. amylovora. Thus, application of FVG, either through direct application of FVG-producing bacteria or through culture filtrate, is an effective method to inhibit the growth of E. amylovora.

As described in U.S. patent application Ser. No. 12/567,590, incorporated herein by reference in its entirety, FVG, also known as the Germination Arrest Factor (GAF), is a small, hydrophilic molecule that is insoluble or sparingly soluble in organic solvents and reacts with ninhydrin. GAF chromatographs with defined R_{f} values in particular thin-layer chromatography systems, and mutation of any of two genes that have been identified as essential for GAF biosynthesis and secretion results in both loss of the biological activity associated with GAF and loss of one particular ninhydrin-positive band visible in these TLC separations. Purification and analysis of the compound responsible for GAF activity has identified GAF as FVG, that is, as 2(R/S)-amino-4-formylaminooxy-but-3-enoic acid (a.k.a., 4-formylaminooxyvinylglycine).

As shown in Example 1, the specific antimicrobial property of FVG was further demonstrated in tests of a number of other Pseudomonas isolates. These results demonstrated that while FVG-producing isolates possess anti-Erwinia activity, Pseudomonas isolates that do not produce FVG are either inactive against Erwinia (e.g., P. fluorescens isolate TDH5 and P. fluorescens strain PfO-1) or active by virtue of the production of other types of antimicrobial compounds. In particular examples, the strain of FVG-producing bacteria is Pseudomonas fluorescens strain isolate WH6 (NRRL# B-30485), AD31 (NRRL# B-30483), AH4 (NRRL#B-30482), E34 (NRRL# B-30481), or WH19 (NRRL# B-30484); all of which were deposited under the conditions of the Budapest Treaty with the ARS Patent Culture Collection on Jun. 13, 2001. In other examples, the strain of FVG-producing bacteria is Pseudomonas fluorescens strain isolate AH10 (NRRL# B-50232), BT1 (NRRL#B-50230), E24 (NRRL# B-50229), TR33 (NRRL# B-50220), TR44 (NRRL# B-50219), TR46 (NRRL# B-50218), A3422A (NRRL# B-50234), ALW38 (NRRL# B-50231), G2Y (NRRL# B-50228), GTR12 (NRRL# B-50227), GTR24 (NRRL# B-50226), GTR40 (NRRL# B-50225), HB14 (NRRL# B-50224), HB26 (NRRL# B-50223), HB32 (NRRL# B-50222), ST22 (NRRL# B-50221), W36 (NRRL# B-50217), or Pseudomonas mucidolens/synxantha A342 (NRRL# B-50236) or TDH40 (NRRL# B-50235), or Enterobacter kobei A3203 (NRRL# B-50233); all of which were deposited, under the conditions of the Budapest Treaty, with the ARS Patent Culture Collection on Jan. 23, 2009.

Based on the ability of FVG and FVG-producing bacteria to inhibit E. amylovora growth, it is likely that direct application of either FVG or FVG-producing bacteria onto plants known to harbor Erwinia will inhibit growth of E. amylovora on the plant. In particular examples, the plant is a member of family Rosaceae. In further examples, the plant is an apple or a pear tree.

### B. AVG

In particular embodiments of the disclosed method, 4-aminoethoxyvinylglycine (AVG, 2-amino-4-aminoethoxy-but-3-enoic acid) can be used to inhibit the growth of E. amylovora, and so can be used as a control agent for fire blight. As discussed above, identification of FVG (2-amino-4-formylaminooxy-but-3-enoic acid, or 4-formylaminooxyvinylglycine) as the compound responsible for GAF activity and the antimicrobial properties disclosed herein, indicated that other vinylglycines, such as AVG, might similarly inhibit E. amylovora growth. While AVG has known antimicrobial properties (see U.S. Pat. No. 3,751,459), its particular efficacy at inhibiting E. amylovora growth has not been characterized. As shown in Example 2, both pure AVG and the AVG-containing commercial preparation ReTain® were effective at inhibiting the growth of E. amylovora. Because AVG is effective as a growth inhibitor of E. amylovora, it is likely that it will also be effective as a control agent against fire blight in blooming plants.

AVG is commercially available, both as a pure compound and as a 15% formulation marketed under the trade name ReTain®. ReTain® is used as a plant growth regulator to inhibit ethylene production in fruit trees (e.g. apple and pear) with ripening fruit in order to extend the growing season, prevent premature fruit drop, maintain fruit firmness, and delay fruit maturation. According to the manufacturer's instructions, ReTain® should be applied approximately four to five weeks prior to harvest and after fruit-set. The length of a growing season, and the time at which a given crop is harvested varies widely, and is dependent on such factors as crop type, average climate, and local weather conditions. Despite this variation, flowers are normally not present in crops four to five weeks prior to harvest. As noted above, the predominant route of E. amylovora infection and crop loss is through blossom infection and blight. Thus, according to current practice, AVG is not applied in a temporal context that would allow for control of E. amylovora infection, and by extension, fire blight disease development. In some examples, AVG is used to control E. amylovora and the spread of fire blight in pome fruit trees by application of AVG from the tight cluster to petal fall stages of development for apple trees and from bud burst to petal fall stages of development for pear trees (available online at web1.msue.msu.edu/fruit/indexbud.htm for a description of the stages of blossom and fruit development for apples and pears).

### C. Non-Vinylglycine Inhibitors of Pyridoxal-Phosphate-Dependent Enzyme Reactions

In particular embodiments, compounds that inhibit pyridoxal-phosphate-dependent enzyme reactions can be used to inhibit the growth of E. amylovora, and thus can be used as control agents for fire blight. Vinylglycines, and in particular AVG, are known to inhibit pyridoxal-phosphate-dependent enzyme reactions. Enzymes that catalyze such reactions are known to include a variety of aminotransferases (enzymes that are important in nitrogen metabolism) and 1-aminocyclopropane-1-carboxylate (ACC) synthase, a key enzyme in the biosynthesis of the plant hormone ethylene. As both FVG and AVG inhibited the growth of E. amylovora, it appeared likely that compounds of different structure but a similar mode of action should be similarly effective. As shown in Example 2, aminooxyacetic acid (AOA), a synthetic inhibitor known to block pyridoxal-phosphate-dependent reactions, was effective at inhibiting the growth of E. amylovora. Because AOA is effective as a growth inhibitor of E. amylovora, it is likely that it will also be effective as a control agent against fire blight in blooming plants.

### D. Structurally-Related Analogs of FVG, AVG, and AOA.

Based on the results with FVG, AVG, and AOA, the ability to inhibit the growth of Erwinia amylovora appears to be a general property of vinylglycines and other types of compounds that share with vinylglycines the ability to inhibit enzyme reactions that are dependent on pyridoxal-phosphate. Structurally-related analogs of these compounds might also be expected to serve as agents for the control of E. amylovora as described below.

A number of structural modifications or derivatives of the specific FVG molecule specifically contemplated for use in the methods and compositions disclosed herein include, without limitation, those of Formulae IA, IB and IC:

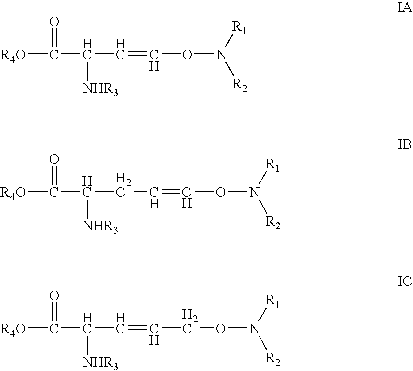

wherein R₁ and R₂ independently are selected from H, optionally substituted lower aliphatic, such as lower alkyl, optionally substituted amino, alkoxy, hydroxy, —COOH, hydroxy alkyl, alkyl amino and the like.

With continued reference to Formulae IA, IB, and IC, R₃ independently is selected from H, and optionally substituted lower aliphatic, such as optionally substituted lower alkyl, acetyl and propanyl. In exemplary embodiments, R₃ is H, and in other embodiments R₃ is lower alkyl, such as methyl.

With continued reference to Formulae IA, IB, and IC, R₄ independently is selected from H, and optionally substituted lower aliphatic, such as optionally substituted lower alkyl. In exemplary embodiments, R₄ is H, and in other embodiments R₄ is lower alkyl, such as methyl or ethyl.

Particular examples of compounds having Formulae IA, IB, and IC, wherein R₁, R₂, R₃ and R₄ are as follows:
R₁=CH₂CH₃, R₂=H, CH₃, R₃=H, CH₃, COCH₃, R₄=H, CH₃, CH₃CH₂;
R₁=CH₂CH₂CH₃, R₂=H, CH₃, R₃=H, CH₃, COCH₃, R₄=H, CH₃, CH₃CH₂;
R₁=CH₂CH₂CH₂CH₃, R₂=H, CH₃, R₃=H, CH₃, COCH₃, R₄=H, CH₃, CH₃CH₂;
R₁=CH₂NH₂, R₂=H, CH₃, R₃=H, CH₃, COCH₃, R₄=H, CH₃, CH₃CH₂;
R₁=CH₂CH₂NH₂, R₂=H, CH₃, R₃=H, CH₃, COCH₃, R₄=H, CH₃, CH₃CH₂;
R₁=CH₂CH₂CH₂NH₂, R₂=H, CH₃, R₃=H, CH₃, COCH₃, R₄=H, CH₃, CH₃CH₂;
R₁=CH₂CH(CH₃)NH₂, R₂=H, CH₃, R₃=H, CH₃, COCH₃, R₄=H, CH₃, CH₃CH₂;
R₁=CH₂CH(NH₂)CH₂NH₂, R₂=H, CH₃, R₃=H, CH₃, COCH₃, R₄=H, CH₃, CH₃CH₂;
R₁=CH₂OH, R₂=H, CH₃, R₃=H, CH₃, COCH₃, R₄=H, CH₃, CH₃CH₂;
R₁=CH₂CH₂OH, R₂=H, CH₃, R₃=H, CH₃, COCH₃, R₄=H, CH₃, CH₃CH₂;
R₁=CH₂CH₂CH₂OH, R₂=H, CH₃, R₃=H, CH₃, COCH₃, R₄=H, CH₃, CH₃CH₂;
R₁=CH₂CH(CH₃)OH, R₂=H, CH₃, R₃=H, CH₃, COCH₃, R₄=H, CH₃, CH₃CH₂;
R₁=CH₂CH(OH)CH₂OH, R₂=H, CH₃, R₃=H, CH₃, COCH₃, R₄=H, CH₃, CH₃CH₂;
R₁=CH₂CHO, R₂=H, CH₃, R₃=H, CH₃, COCH₃, R₄=H, CH₃, CH₃CH₂;
R₁=CH₂CH₂CHO, R₂=H, CH₃, R₃=H, CH₃, COCH₃, R₄=H, CH₃, CH₃CH₂;
R₁=CH₂COOH, R₂=H, CH₃, R₃=H, CH₃, COCH₃, R₄=H, CH₃, CH₃CH₂;
R₁=CH₂CH₂COOH, R₂=H, CH₃, R₃=H, CH₃, COCH₃, R₄=H, CH₃, CH₃CH₂;
R₁=COOH, R₂=H, CH₃, R₃=H, CH₃, COCH₃, R₄=H, CH₃, CH₃CH₂; or
R₁=COOCH₃, R₂=H, CH₃, R₃=H, CH₃, COCH₃, R₄=H, CH₃, CH₃CH₂.

Additional structural modifications or derivatives of the FVG molecule specifically contemplated for use in the methods and compositions disclosed herein include, without limitation, those of Formulae IIA, IIB, and IIC:

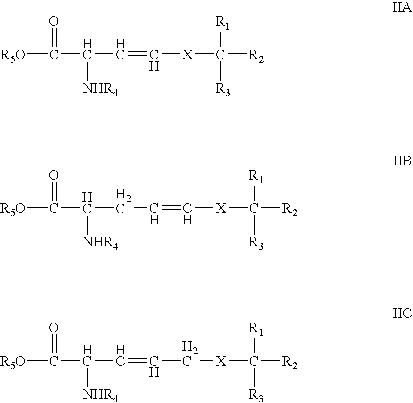

wherein the variable group X typically is selected from O, S and optionally substituted amino, and R₁, R₂ and R₃ independently are selected from H, optionally substituted lower aliphatic, such as lower alkyl, optionally substituted amino, alkoxy, hydroxy, —COOH, —NHCOOH, hydroxy alkyl, alkyl amino and the like.

With continued reference to Formulae IIA, IIB, and IIC, R₄ independently is selected from H, and optionally substituted lower aliphatic, such as optionally substituted lower alkyl, acetyl and propanyl. In exemplary embodiments, R₄ is H, and in other embodiments R₄ is lower alkyl, such as methyl.

With continued reference to Formulae IIA, IIB, and IIC, R₅ independently is selected from H, and optionally substituted lower aliphatic, such as optionally substituted lower alkyl. In exemplary embodiments, R₅ is H, and in other embodiments R₅ is lower alkyl, such as methyl or ethyl.

Particular examples of compounds have Formulae IIA, IIB, and IIC wherein X, R₁, R₂, R₃, R₄ and R₅ are as follows:
X=NH, NHCH₃, S; R₁=CH(CH₃)CH₃, R₂=R₃=H, R₄=H, CH₃, COCH₃, R₅=H, CH₃;
X=O, NH, S; R₁=CH₃, R₂=H, CH₃, R₃=H, R₄=H, CH₃, COCH₃, R₅=H, CH₃;
X=O, NH, S; R₁=CH₂CH₃, R₂=H, CH₃, R₃=H, R₄=H, CH₃, COCH₃, R₅=H, CH₃;
X=O, NH, S; R₁=CH₂CH₂CH₃, R₂=H, CH₃, R₃=H, R₄=H, CH₃, COCH₃, R₅=H, CH₃;
X=O, NH, S; R₁=CH₂NH₂, R₂=H, CH₃, R₃=H, R₄=H, CH₃, COCH₃, R₅=H, CH₃;
X=O, NH, S; R₁=CH₂CH₂NH₂, R₂=H, CH₃, R₃=H, R₄=H, CH₃, COCH₃, R₅=H, CH₃;
X=O, NH, S; R₁=CH(CH₃)NH₂, R₂=H, CH₃, R₃=H, R₄=H, CH₃, COCH₃, R₅=H, CH₃;
X=O, NH, S; R₁=CH(NH₂)CH₂NH₂, R₂=H, CH₃, R₃=H, R₄=H, CH₃, COCH₃, R₅=H, CH₃;
X=O, NH, S; R₁=CH₂OH, R₂=H, CH₃, R₃=H, R₄=H, CH₃, COCH₃, R₅=H, CH₃;
X=O, NH, S; R₁=CH₂CH₂OH, R₂=H, CH₃, R₃=H, R₄=H, CH₃, COCH₃, R₅=H, CH₃;
X=O, NH, S; R₁=CH(CH₃)OH, R₂=H, CH₃, R₃=H, R₄=H, CH₃, COCH₃, R₅=H, CH₃;
X=O, NH, S; R₁=CH(OH)CH₂OH, R₂=H, CH₃, R₃=H, R₄=H, CH₃, COCH₃, R₅=H, CH₃;
X=O, NH, S; R₁=CHO, R₂=H, CH₃, R₃=H, R₄=H, CH₃, COCH₃, R₅=H, CH₃;
X=O, NH, S; R₁=CH₂CHO, R₂=H, CH₃, R₃=H, R₄=H, CH₃, COCH₃, R₅=H, CH₃;
X=O, NH, S; R₁=CH₂COOH, R₂=H, CH₃, R₃=H, R₄=H, CH₃, COCH₃, R₅=H, CH₃;
X=O, NH, S; R₁=COOH, R₂=H, CH₃, R₃=H, R₄=H, CH₃, COCH₃, R₅=H, CH₃;
X=O, NH, S; R₁=NH₂, R₂=H, CH₃, R₃=H, R₄=H, CH₃, COCH₃, R₅=H, CH₃;
X=O, NH, S; R₁=OH, R₂=H, CH₃, R₃=H, R₄=H, CH₃, COCH₃, R₅=H, CH₃; or X=N; R₁=O, R₂=OH, R₃=H, R₄=H, CH₃, COCH₃, R₅=H, CH₃.

Still additional structural modifications of the FVG molecule specifically contemplated for use in the methods and compositions disclosed herein include, without limitation, those of Formula III:

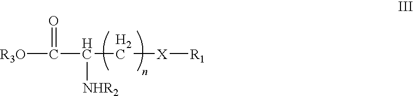

wherein the variable group X typically is selected from O, S and optionally substituted amino, and R₁ independently is selected from H, optionally substituted lower aliphatic, such as lower alkyl, optionally substituted amino, alkoxy, hydroxy, —NHCOOH, hydroxy alkyl, alkyl amino and the like, and wherein n=1, 2, 3, 4, or 5.

With continued reference to Formula III, R₂ independently is selected from H, and optionally substituted lower aliphatic, such as optionally substituted lower alkyl, acetyl and propanyl. In exemplary embodiments, R₂ is H, and in other embodiments R₂ is lower alkyl, such as methyl.

With continued reference to Formula III, R₃ independently is selected from H, and optionally substituted lower aliphatic, such as optionally substituted lower alkyl. In exemplary embodiments, R₃ is H, and in other embodiments R₃ is lower alkyl, such as methyl or ethyl.

In particular examples, the structural modifications of the GAF molecule used in the present methods and compositions include those of Formula III, wherein n=1 and R₁, R₂, R₃ and X are:
X=O, NH, S; R₁=CH₂CH₂CH₂NH₂, R₂=H, CH₃, COCH₃, R₃=H, CH₃;
X=O, NH, S; R₁=CH₂CH(NH₂)CH₂NH₂, R₂=H, CH₃, COCH₃, R₃=H, CH₃;
X=O, NH, S; R₁=CH₂CH(OH)CH₂OH, R₂=H, CH₃, COCH₃, R₃=H, CH₃;
X=O, NH, S; R₁=CH₂CHO, R₂=H, CH₃, COCH₃, R₃=H, CH₃;
X=O, NH, S; R₁=CH₂CH₂CHO, R₂=H, CH₃, COCH₃, R₃=H, CH₃;
X=O, NH; R₁=CH₂COOH, R₂=H, CH₃, COCH₃, R₃=H, CH₃;
X=O, NH, S; R₁=CH₂CH₂COOH, R₂=H, CH₃, COCH₃, R₃=H, CH₃; or
X=O; R₁=NH₂, R₂=H, CH₃, COCH₃, R₃=H, CH₃;
   and wherein n=2, R₁, R₂, R₃ and X are:
X=NH; R₁=OH, OCH₃, R₂=H, CH₃, COCH₃, R₃=H, CH₃;
X=O; R₁=H, NH₂, R₂=H, CH₃, COCH₃, R₃=H, CH₃;
X=O, NH; R₁=CH₃, R₂=H, CH₃, COCH₃, R₃=H, CH₃;
X=O, NH, S; R₁=CH₂CH₃, R₂=H, CH₃, COCH₃, R₃=H, CH₃;
X=O, NH, S; R₁=CH₂CH₂CH₃, R₂=H, CH₃, COCH₃, R₃=H, CH₃;
X=O, NH, S; R₁=CH₂CH₂CH₂CH₃, R₂=H, CH₃, COCH₃, R₃=H, CH₃;
X=O, NH, S; R₁=CH₂CH(CH₃)CH₃, R₂=H, CH₃, COCH₃, R₃=H, CH₃;
X=O, NH, S; R₁=CH₂NH₂, R₂=H, CH₃, COCH₃, R₃=H, CH₃;
X=O, NH; S; R₁=CH₂CH₂NH₂, R₂=H, CH₃, COCH₃, R₃=H, CH₃;
X=O, NH, S; R₁=CH₂CH₂CH₂NH₂, R₂=H, CH₃, COCH₃, R₃=H, CH₃;
X=O, NH, S; R₁=CH₂CH(CH₃)NH₂, R₂=H, CH₃, COCH₃, R₃=H, CH₃;
X=O, NH, S; R₁=CH₂CH(NH₂)CH₂NH₂, R₂=H, CH₃, COCH₃, R₃=H, CH₃;
X=O, NH, S; R₁=CH₂OH, R₂=H, CH₃, COCH₃, R₃=H, CH₃;
X=O, NH, S; R₁=CH₂CH₂OH, R₂=H, CH₃, COCH₃, R₃=H, CH₃;
X=O, NH, S; R₁=CH₂CH₂CH₂OH, R₂=H, CH₃, COCH₃, R₃=H, CH₃;
X=O, NH, S; R₁=CH₂CH(CH₃)OH, R₂=H, CH₃, COCH₃, R₃=H, CH₃;
X=O, NH, S; R₁=CH₂CH(OH)CH₂OH, R₂=H, CH₃, COCH₃, R₃=H, CH₃;
X=O, NH, S; R₁=CH₂CHO, R₂=H, CH₃, COCH₃, R₃=H, CH₃;
X=O, NH, S; R₁=CH₂CH₂CHO, R₂=H, CH₃, COCH₃, R₃=H, CH₃;
X=O, NH, S; R₁=CH₂COOH, R₂=H, CH₃, COCH₃, R₃=H, CH₃;
X=O, NH, S; R₁=CH₂CH₂COOH, R₂=H, CH₃, COCH₃, R₃=H, CH₃;
X=O; R₁=NHCOCH₃, R₂=H, CH₃, COCH₃, R₃=H, CH₃;
X=O; R₁=NH(COCH₃)₂, R₂=H, CH₃, COCH₃, R₃=H, CH₃;
X=O; R₁=NHCOOH, R₂=H, CH₃, COCH₃, R₃=H, CH₃;
X=O; R₁=NHCOOCH₃, R₂=H, CH₃, COCH₃, R₃=H, CH₃; or
X=O; R₁=NHCHO, R₂=H, CH₃, COCH₃, R₃=H, CH₃.

Still other structural modifications of the GAF molecule for use in the present methods and compositions include those of Formula IV:

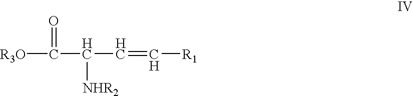

wherein
R₁=NHOH, R₂=H, CH₃, COCH₃, R₃=H, CH₃, CH₃CH₂;
R₁=NHOCH₃, R₂=H, CH₃, COCH₃, R₃=H, CH₃, CH₃CH₂;
R₁=OCH₂CH₂NH₂, R₂=H, CH₃, COCH₃, R₃=H, CH₃, CH₃CH₂;
R₁=OCH₃, R₂=H, CH₃, COCH₃, R₃=H, CH, CH₃CH₂₃;
R₁=ONH₂, R₂=H, CH₃, COCH₃, R₃=H, CH₃, CH₃CH₂;
R₁=CH₂PO₃²⁻, R₂=H, CH₃, COCH₃, R₃=H, CH₃, CH₃CH₂;
R₁=CH₂CH₂CO₂H, R₂=H, CH₃, COCH₃, R₃=H, CH₃, CH₃CH₂;
R₁=CHCH₂, R₂=H, CH₃, COCH₃, R₃=H, CH₃, CH₃CH₂;
R₁=CH₂NHCONH₂, R₂=H, CH₃, COCH₃, R₃=H, CH₃, CH₃CH₂;

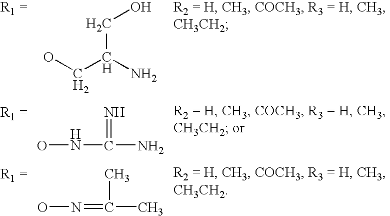

Additional compounds according to Formula IV suitable for use in the present methods and compositions include alkyloxy and aryloxy vinylglycines, including those wherein
R₁=OCH₂CH(CH₃)CH₃, R₂=H, CH₃, COCH₃, R₃=H, CH₃, CH₃CH₂;

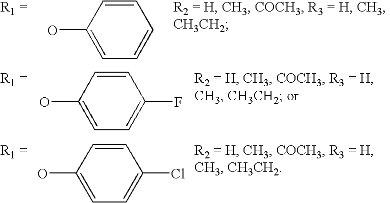

Still other compounds of Formula IV are thiovinylglycines, wherein R₁ is a substituted thiol moiety, such as a lower alkyl substituted thiol moiety, for example wherein R₁=S—CH₃, R₂=H, CH₃, COCH₃, R₃=H, CH₃, CH₃CH₂.

In one embodiment, suitable structural modifications of the FVG molecule for use in the present methods and compositions include those of the formulas

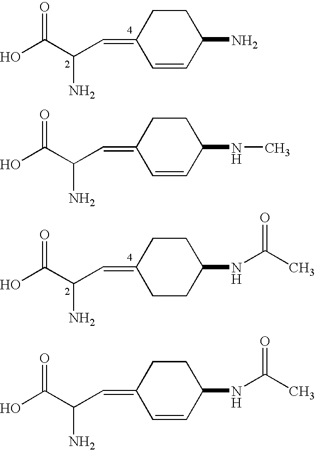

Additional examples of structural modifications of the FVG molecule include, without limitation, β,γ-disubstituted vinylic amino acids of the general formula V

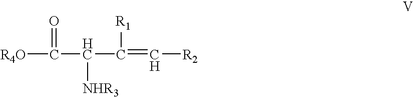

wherein the substituents R₁ and R₂ independently are selected from optionally substituted lower aliphatic, such as lower alkyl, and halo, in particular chloro and bromo, R₃ is selected from H, lower alkyl, acetyl and propanyl, and R₄ is selected from H and lower alkyl including those wherein:
R₁=CH₂OH; R₂=CH₃, R₃=H, CH₃, COCH₃, R₄=H, CH₃;
R₁=CHO; R₂=CH₃, R₃=H, CH₃, COCH₃, R₄=H, CH₃; or
R₁=Cl; R₂=CO₂H, R₃=H, CH₃, COCH₃, R₄=H, CH₃.

Other organic acids, for example those that share with vinylglycines the ability to act as inhibitors of pyridoxal-phosphate-dependent enzyme reactions, including reactions catalyzed by aminotransferases and the reaction catalyzed by ACC synthase in ethylene biosynthesis, also may be useful in the present methods and compositions either alone or in combination with FVG or other molecules structurally related to FVG. Examples of such organic acids include but are not limited to
H₂N—O—CH₂—COOH: 2-Aminooxyacetic acid;
OHC—NH—O—CH₂—COOH: 2-Formylaminooxyacetic acid;
H₂N—CO—NH—O—CH₂—COOH: 2-Ureidooxyacetic acid;
H₂N—CH₂—CH₂—O—CH₂—COOH: 2-Aminoethoxyacetic Acid;
H₂N—O—CH₂—CH₂—COOH: 3-Aminooxypropanoic acid;
OHC—NH—O—CH₂—CH₂—COOH: 3-Formylaminooxypropanoic acid;
H₂N—CO—NH—O—CH₂—CH₂—COOH: 3-Ureidooxypropanoic acid;
H₂N—CH₂—CH₂—O—CH₂—CH₂—COOH: 3-Aminoethoxypropanoic acid;
H₂N—O—CH₂—CH₂—CH₂—COOH: 4-Aminooxybutanoic acid;
OHC—NH—O—CH₂—CH₂—CH₂—COOH: 4-Formylaminooxybutanoic acid;
H₂N—CO—NH—O—CH₂—CH₂—COOH: 4-Ureidooxybutanoic acid;
H₂N—CH₂—CH₂—O—CH₂—CH₂—CH₂—COOH: 4-Aminoethoxybutanoic acid;

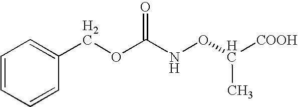

N-benzyloxycarbonyl-L-2-aminooxypropanoic acid;

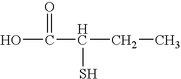

2-mercaptobutanoic acid;
   and
cyclic amino acids of the formula VI:

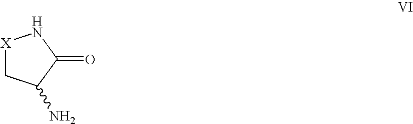

for example those wherein
X=O: Cycloserine;
X=S: (R/S)-4-Amino-3-isothiazolidinone; and
X=N: Azacycloserine {(R/S)-4-Amino-3-Pyrazolidinone}.

Additional structural modifications of the FVG molecule will be apparent to those of ordinary skill in the art upon consideration of the formulas above and further in view of the entirety of the present disclosure.

### E. Chemical Formulations

FVG (2-amino-4-formylaminooxybut-3-enoic acid) and the structurally and mechanistically related compounds of the present disclosure can be combined with appropriate solvents or surfactants to form a product called a formulation. Formulations enable the uniform distribution of a relatively small amount of FVG, or structurally related compounds of similar biological activity, over a comparatively large area. In addition to providing the user with a form of FVG that is easy to handle, formulating FVG can enhance its antimicrobial effects, improve its shelf-life, and protect it from adverse environmental conditions while in storage or transit.

The primary kinds of formulations are: solutions, soluble powders, emulsifiable concentrates, wettable powders, liquid flowables, dry flowables, water-dispersible granules, granules, pellets, and pesta. Formulations vary according to the solubility of the active ingredient in water, oil and organic solvents, and the manner the formulation is applied (i.e., dispersed in a carrier, such as water, or applied as a dry formulation).

Solution formulations are designed for those active ingredients that dissolve readily in water. The formulation is a liquid and consists of the active ingredient and additives. Suitable liquid carriers, such as solvents, may be organic or inorganic. Water is one example of an inorganic liquid carrier. Organic liquid carriers include vegetable oils and epoxidized vegetable oils, such as rape seed oil, castor oil, coconut oil, soybean oil and epoxidized rape seed oil, epoxidized castor oil, epoxidized coconut oil, epoxidized soybean oil, and other essential oils. Other organic liquid carriers include silicone oils, aromatic hydrocarbons, and partially hydrogenated aromatic hydrocarbons, such as alkylbenzenes containing 8 to 12 carbon atoms, including xylene mixtures, alkylated naphthalenes, or tetrahydronaphthalene. Aliphatic or cycloaliphatic hydrocarbons, such as paraffins or cyclohexane, and alcohols, such as ethanol, propanol or butanol, also are suitable organic carriers. Gums, resins, and rosins used in forest products applications and naval stores (and their derivatives) also may be used. Additionally, glycols, including ethers and esters, such as propylene glycol, dipropylene glycol ether, diethylene glycol, 2-methoxyethanol, and 2-ethoxyethanol, and ketones, such as cyclohexanone, isophorone, and diacetone alcohol may be used. Strongly polar organic solvents include N-methylpyrrolid-2-one, dimethyl sulfoxide, and N,N-dimethylformamide.

Typical liquid diluents and solvents are described in Marsden, Solvents Guide, 2nd Ed., Interscience, NY, 1950. Solubility under 0.1% is preferred for suspension concentrates; solution concentrates are preferably stable against phase separation at 0° C. McCutcheon's Detergents and Emulsifiers Annual, Allured Publ. Corp., Ridgewood, N.J., as well as Sisely and Wood, Encyclopedia of Surface Active Agents, Chemical Publ., Co., Inc., NY 1964, list surfactants and recommended uses.

Soluble powder formulations are similar to solutions in that, when mixed with water, they dissolve readily and form a true solution. Soluble powder formulations are dry and include the active ingredient and additives. When thoroughly mixed, no further agitation is necessary to keep the active ingredient dissolved in solution.

Emulsifiable concentrate formulations are liquids that contain the active ingredient, one or more solvents, and an emulsifier that allows mixing with water. Formulations of this type are highly concentrated, relatively inexpensive per pound of active ingredient, and easy to handle, transport, and store. In addition, they require little agitation (will not settle out or separate) and are not abrasive to machinery or spraying equipment. Formulations of this type may, however, have greater phytotoxicity than other formulations, and they are subject to over- or under-dosing through mixing or calibration errors. In addition, these types of formulations are more easily absorbed through the skin of humans or animals, and contain solvents that may cause deterioration of rubber or plastic hoses and pump parts.

Wettable powders are dry, finely ground formulations in which the active ingredient is combined with a finely ground carrier (usually mineral clay), along with other ingredients to enhance the ability of the powder to suspend in water. Generally, the powder is mixed with water for application. Wettable powders are some of the most widely used formulations and offer low cost and ease of storage, transport, and handling; lower phytotoxicity potential than emulsifiable concentrates and other liquid formulations; and less skin and eye absorption hazard than emulsifiable concentrates and other liquid formulations. Some disadvantages are that they require constant and thorough agitation in the spray tank, are abrasive to pumps and nozzles (causing premature wear), may produce visible residues on plant and soil surfaces, and can create an inhalation hazard to the applicator while handling (pouring and mixing) the concentrated powder. Typical solid diluents are described in Watkins et al., Handbook of Insecticide Dust Diluents and Carriers, 2nd Ed., Dorland Books, Caldwell, N.J. The more absorptive diluents are preferred for wettable powders and the denser ones for dusts.

Liquid flowable formulations are made up of finely ground active ingredient suspended in a liquid. Flowables generally are mixed with water for application, are easily handled and applied, and seldom clog nozzles. Some of their disadvantages are that they may leave a visible residue on plant and soil surfaces, and typically require constant and thorough agitation to remain in suspension.

Dry flowable and water-dispersible granule formulations are much like wettable powders except that the active ingredient is formulated on a large particle (granule) instead of onto a ground powder. This type of formulation offers essentially the same advantages and disadvantages as wettable powder formulations. However, these formulations generally are more easily mixed and measured than wettable powders. Because they create less dust when handling, they cause less inhalation hazard to the applicator during pouring and mixing.

Granule and pellet formulations are preferred for some uses. In a granule or pellet formulation, the active ingredient is formulated onto large particles (granules or pellets). The primary advantages of this type of formulation are that the formulation is ready to use with simple application equipment, and the drift potential is low because the particles are large and settle quickly. The disadvantages of these formulations are that they do not adhere well to foliage and may require the use of additives such as sticker-spreaders.

Granulated materials of inorganic or organic nature may be used in formulating granules, pellets, or pesta such as dolomite or pulverized plant residues. Suitable porous granulated adsorptive carriers include pumice, broken brick, sepiolite, and bentonite. Additionally, nonsorbent carriers, such as sand, may be used. Some solid carriers are biodegradable polymers, including biodegradable polymers that are digestible or degrade inside an animal's body over time.

The methods of making such formulations are well known. Solutions are prepared by simply mixing the ingredients. Fine, solid compositions are made by blending and, usually, grinding, as in a hammer or fluid energy mill. Suspensions are prepared by wet-milling (see, for example, U.S. Pat. No. 3,060,084). Granules and pellets may be made by spraying the active material upon preformed granular carriers or by agglomeration techniques. See J. E. Browning, “Agglomeration”, Chemical Engineering, Dec. 4, 1967, p 147, and Perry's Chemical Engineer's Handbook, 4th Ed., McGraw-Hill, NY, 1963, pp. 8-59. For further information regarding the art of formulation, see, for example: U.S. Pat. No. 3,235,361, U.S. Pat. No. 3,309,192, U.S. Pat. No. 2,891,855, Klingman, Weed Control as a Science, John Wiley & Sons, Inc., New York, 1961 pp. 81-96, and Fryer and Evans, Weed Control Handbook, 5th Edn. Blackwell Scientific Publications, Oxford, 1968, pp. 101-103.

In selecting a formulation for use as a fire blight control agent, the following considerations may be weighed: 1) how the formulation will affect efficacy of the agent and influence phytotoxicity or other undesirable side-effects of treatment, 2) how the formulation will influence the compatibility of other crop protection chemicals, 3) what application machinery are available and most suited for the job, 4) how the formulation will affect the life of the application equipment, 5) whether the application equipment is designed for applying a particular formulation, and 6) concerns about safety for the applicator and other people.

The concentration of a compound, such as FVG, which serves as an active ingredient, may vary according to particular compositions and applications. In some embodiments of the disclosure, the percentage by weight of the active ingredient will be from about 0.1% to about 90%, for example to give final FVG concentrations equivalent to, or greater than, that in Pseudomonas fluorescens WH6 culture filtrate. A suitable amount for a particular application may be determined using bioassays for the inhibition of E. amylovora growth. Higher concentrations are usually employed for commercial purposes or products during manufacture, shipment, or storage; such embodiments have concentrations at least about 10%, or from about 25% to about 90% by weight. Prior to use, a highly concentrated formulation may be diluted to a concentration appropriate for the intended use, such as from about 0.1% to 10%, or from about 1% to 5%.

### F. Bacterial Application

Based on the disclosure herein, it can now be appreciated that Pseudomonas bacteria capable of producing FVG, FVG-structural variants, or other types of vinylglycines, can be used to inhibit the growth of E. amylovora or as an agent to control fire blight.

As will be understood by those with skill in the art, with reference to this disclosure, in order for bacterial strains which produce FVG (2-amino-4-formylaminooxy-but-3-enoic acid) or the FVG-variants which inhibit growth of E. amylovora and can control fire blight to be grown, cultured, or used in accordance with the embodiments of the present invention, these bacterial strains will be grown in a medium suitable to produce a biocontrol composition or formulation. The term “suitable medium” or “acceptable medium” is meant to include any liquid, semi-liquid, or solid substrate which allows FVG-producing bacterial strains or bacterial strains that produce other FVG structural variants to grow, or to remain viable, or both grow and remain viable, for example during storage. Furthermore, these bacterial strains may be formulated as indicated below prior to use. Such formulations are also considered suitable or acceptable media in the context of the present invention. Preferably, the formulation permits an effective amount of one or more FVG-producing bacterial strain to remain viable prior to, and after, being applied to a crop. More preferably, the medium, formulation, or both medium and formulation permits FVG-producing bacterial strains or bacterial strains that produce FVG structural variants that inhibit growth of E. amylovora to remain viable for about 1 to 3 months following application of the bacteria to the target plants.

The present invention also contemplates growing FVG-producing bacterial strains or bacterial strains that produce FVG structural variants that inhibit growth of E. amylovora in various types of media, including, but not limited to, Pseudomonas Minimal Salts (PMS) medium with or without additional supplements, 925 minimal medium, nutrient broth, M9 medium and REC medium. The present invention also contemplates formulations of the bacteria in pesta, peat prills, vermiculite, clay, starches, wheat straw (see for example Connick et al., 1991; Fravel, Connick and Lewis, 1998. Formulation of microorganisms to control plant diseases. p. 187-202 In: H. D. Burges (Ed.), Formulation of Microbial Biopesticides, Kluwer Academic Publishers, Dordrecht, The Netherlands.; Quimby et al., Biocontrol Science and Technology 9:5-8, 1999; U.S. Pat. Nos. 5,074,902 and 5,358,863; and International Publication WO 98/05213), or any combination or variant thereof, provided that the formulation allows the bacterial strain that produces FVG or other vinylglycines to remain viable. The biocontrol agent may also be applied to the surface of the plant in a suitable formulation or composition as would be known to one of skill in the art.

### G. Inactive Ingredients in Formulations

In some embodiments of the disclosure, inactive ingredients (that is, adjuvants) are added to anti-Erwinia formulations to improve the performance of the formulation. For example, in one embodiment of the disclosure, an FVG molecule is formulated with a surfactant. A surfactant (surface active agent) is a type of adjuvant designed to improve the dispersing/emulsifying, absorbing, spreading, sticking and/or pest-penetrating properties of the spray mixture. Surfactants can be divided into the following five groupings: 1) non-ionic surfactants, 2) crop oil concentrates, 3) nitrogen-surfactant blends, 4) esterified seed oils, and 5) organo-silicones.

Suitable surfactants may be nonionic, cationic, or anionic, depending on the nature of the compound used as an active ingredient. Surfactants may be mixed together in some embodiments of the disclosure. Nonionic surfactants include polyglycol ether derivatives of aliphatic or cycloaliphatic alcohols, saturated or unsaturated fatty acids and alkylphenols. Fatty acid esters of polyoxyethylene sorbitan, such as polyoxyethylene sorbitan trioleate, also are suitable nonionic surfactants. Other suitable nonionic surfactants include water-soluble polyadducts of polyethylene oxide with polypropylene glycol, ethylenediaminopolypropylene glycol and alkylpolypropylene glycol. Particular nonionic surfactants include nonylphenol polyethoxyethanols, polyethoxylated castor oil, polyadducts of polypropylene and polyethylene oxide, tributylphenol polyethoxylate, polyethylene glycol and octylphenol polyethoxylate. Cationic surfactants include quaternary ammonium salts carrying, as N-substituents, an 8 to 22 carbon straight or branched chain alkyl radical.

The quaternary ammonium salts carrying may include additional substituents, such as unsubstituted or halogenated lower alkyl, benzyl, or hydroxy-lower alkyl radicals. Some such salts exist in the form of halides, methyl sulfates, and ethyl sulfates. Particular salts include stearyldimethylammonium chloride and benzyl bis(2-chloroethyl)ethylammonium bromide.

Suitable anionic surfactants may be water-soluble soaps as well as water-soluble synthetic surface-active compounds. Suitable soaps include alkali metal salts, alkaline earth metal salts, and unsubstituted or substituted ammonium salts of higher fatty acids. Particular soaps include the sodium or potassium salts of oleic or stearic acid, or of natural fatty acid mixtures. Synthetic anionic surfactants include fatty sulfonates, fatty sulfates, sulfonated benzimidazole derivatives, and alkylarylsulfonates. Particular synthetic anionic surfactants include the sodium or calcium salt of ligninsulfonic acid, of dodecyl sulfate, or of a mixture of fatty alcohol sulfates obtained from natural fatty acids. Additional examples include alkylarylsulfonates, such as sodium or calcium salts of dodecylbenzenesulfonic acid, or dibutylnaphthalenesulfonic acid. Corresponding phosphates for such anionic surfactants are also suitable.

Other adjuvants include carriers and additives, for example, wetting agents, such as anionic, cationic, nonionic, and amphoteric surfactants, buffers, stabilizers, preservatives, antioxidants, extenders, solvents, emulsifiers, invert emulsifiers, spreaders, stickers, penetrants, foaming agents, anti-foaming agents, thickeners, safeners, compatibility agents, crop oil concentrates, viscosity regulators, binders, tackers, drift control agents, or other chemical agents, such as fertilizers, antibiotics, fungicides, nematicides, or pesticides. Such carriers and additives may be used in solid, liquid, gas, or gel form, depending on the embodiment and its intended application.

### VI. Kits

In some embodiments of the disclosure, the active compounds described herein, including for instance FVG or compounds that are structurally or mechanistically related to FVG, are embodied in an acceptable carrier and stored within a container capable of storing the composition for its shelf life. The container may be made of any suitable material such as plastic or other polymer, glass, metal, or the like. In some embodiments of the disclosure, printed instructions and/or a printed label indicating that the composition may be used to inhibit the growth of E. amylovora or control fire blight are associated with this container. In certain examples, the instructions and/or label provides information regarding the use of the composition for antimicrobial purposes, and is associated with the container by being adhered to the container, or accompanying the container in a package. In particular examples, the instructions specify the method and rate of application, dilution protocols, use precautions, and the like. Additionally, the container may include a feature or device for applying the composition to the plant surface or locus to be treated. For example, if the article of manufacture includes a liquid composition, the feature or device may be a hand-operated, motorized, or pressurized pressure-driven sprayer.

In some embodiments, for large-scale applications, a kit of the present disclosure may include a drum, whereas for household kits for inhibition of E. amylovora or control of fire blight, FVG or compounds that are structurally or mechanistically related to FVG may be provided in a can or bottle. In other embodiments, FVG or structurally or mechanistically-related compounds are provided as small scale, highly purified material for experimental use in understanding E. amylovora pathogenic processes.

In some embodiments, FVG-producing bacteria or bacterial strains that produce other vinylglycine molecules or mixtures of these with or without other bacterial strains that produce other types of agents with inhibitory effects on E. amylovora may be packaged as a kit consisting of an appropriate container containing the bacteria in various formulations to preserve shelf-life and ensure survival in the field together with instructions for the application and appropriate use of these bacterial preparations for control of fire blight.

The following examples are provided to illustrate certain particular features and/or embodiments. These examples should not be construed to limit the invention to the particular features or embodiments described.

### EXAMPLES

### Example 1

### Inhibitory Effects of Formylaminooxyvinylglycine (FVG) and FVG-Producing Pseudomonas Isolates on Erwinia amylovora

This example shows the inhibitory effects of P. fluorescens strain WH6 and WH6 culture filtrate on the growth of E. amylovora and identifies the source of this inhibition in the capability of bacteria to produce FVG. Additionally, this example shows that other Pseudomonas strains and bacterial species that produce FVG, and culture filtrate from such strains, also inhibit E. amylovora growth. Lastly, this example shows that the antimicrobial property of FVG is specific against only a small subset of bacterial species assayed.

### A. Materials and Methods

Origin, Taxonomic Identification, and Deposit of Reference Cultures of Pseudomonas Isolates: Pseudomonas isolates were classified by fatty acid methyl ester (FAME) analyses and DNA sequencing of a 400+ by region of the 16S-rRNA gene (performed by Microcheck, Inc., Northfield, Vt.). The reported taxonomic identities are based on Microcheck databases as of February, 2008.

Isolates identified as GAF (FVG)-producing bacteria were cultured, lyophilized, and submitted to the ARS Patent Culture Collection for deposit under the conditions of the Budapest Treaty on the International Recognition of the Deposit of Microorganisms for the Purpose of Patent Procedures. P. fluorescens isolates WH6 (NRRL# B-30485), AD31 (NRRL# B-30483), AH4 (NRRL#B-30482), E34 (NRRL# B-30481), and WH19 (NRRL# B-30484) were deposited on Jun. 13, 2001. The date of deposit for P. fluorescens isolates AH10 (NRRL# B-50232), BT1 (NRRL#B-50230), E24 (NRRL# B-50229), TR33 (NRRL# B-50220), TR44 (NRRL# B-50219), TR46 (NRRL# B-50218), A3422A (NRRL# B-50234), ALW38 (NRRL# B-50231), G2Y (NRRL# B-50228), GTR12 (NRRL# B-50227), GTR24 (NRRL# B-50226), GTR40 (NRRL# B-50225), HB14 (NRRL# B-50224), HB26 (NRRL# B-50223), HB32 (NRRL# B-50222), ST22 (NRRL# B-50221), W36 (NRRL# B-50217), and Pseudomonas mucidolens/synxantha A342 (NRRL# B-50236) and TDH40 (NRRL# B-50235), and Enterobacter kobei A3203 (NRRL# B-50233) was Jan. 23, 2009.

Preparation of Culture Filtrates from Pseudomonas fluorescens WH6, Mutant Lines of WH6, and Various Other P. fluorescens Isolates and Strains: P. fluorescens WH6, mutant cell lines derived from WH6 as described in U.S. Patent Publication 2006/0147438, and the various other P. fluorescens isolates and strains used here for the preparation of culture filtrates were taken from stocks originally derived from cells grown in Pseudomonas Minimal Salts Medium (PMS Medium, described below) and stored in 50% glycerol in cryovials at −60° C. For culture filtrate preparation, aliquots (10 μL) of these cryovial preparations were inoculated into Wheaton bottles half-filled with Pseudomonas Minimal Salts Medium (PMS Medium, described below). The tops of the bottles were loosely capped and secured with tape. The bottles were placed on a rotary shaker (200 rpm) in a 27° C. chamber and allowed to grow for 7 days prior to harvest. The cultures were then centrifuged (3,000×g, 15 minutes), and the supernatants were passed through bacteriological filters (Millipore GP Express Steritop, 0.22 μM pore size). The resulting sterile culture filtrates were stored at 4° C.

The PMS medium used for this purpose was based on that developed by Gasson (Gasson, Applied and Environmental Microbiology, 39:25-29, 1980) as modified by Bolton (Bolton et al., Plant and Soil, 114: 279-287, 1989). The medium was made as follows: 0.2 grams of potassium chloride, 1.0 grams of ammonium monobasic phosphate (anhydrous), 2.0 grams or 2.3 grams sodium monobasic phosphate (anhydrous or monohydrate respectively), and 4.96 grams of sodium dibasic phosphate (anhydrous) were dissolved in deionized water and made to 1 liter final volume. This solution was autoclaved and then allowed to cool to room temperature. The following stock solutions were filter-sterilized and added to 1 liter of the sterile cooled medium: 2 mL of 20% (w/v) magnesium sulfate (heptahydrate), 20 mL of 10% (w/v) glucose solution, and 2 mL of 1 mM FeCl₃ dissolved in 10 mM HCl.

Standard Poa Bioassay for GAF (FVG) Activity: Bioassays for GAF (FVG) activity were performed as described by Banowetz et al., Biological Control, 46: 380-390, 2008. Seeds of Poa annua (annual bluegrass) were surface sterilized by successive treatments with 50% sulfuric acid (5 minutes) followed by 100% Clorox containing 1% (v/v) Tween 20 (5 minutes). The seeds were stirred vigorously during each treatment and separated from each treatment solution by filtration in a Gooch® crucible. The seeds in the Gooch® crucible were rinsed with copious amounts of sterile deionized water after each treatment. They were then transferred to a sterile Petri dish and allowed to dry and stored for later use or used immediately in a bioassay.

For bioassay of GAF (FVG) activity, the seeds were transferred to sterile 48-well microplates (Corning Costar® Number 3548) containing 200 μL of test solution per well. Three seeds were placed in each well and three wells (9 seeds) were scored for each concentration of each test solution. The progress of germination was scored after 7 days at 20° C. with a daily photoperiod consisting of 8 hours light (50 microEinsteins) and 16 hours dark. Scores were assigned according to criteria summarized in Table 1.

TABLE 1
Poa Bioassay Scoring System

| Score | Description |
|---|---|
| 0 | No visible signs of germination. |
| 0.5 | Seed has imbibed water, and embryo is white. Seed coat has split, and |
| | the coleorhiza may have emerged from the seed coat, but the coleoptile is not yet |
| | visible external to the seed coat. |
| 1.0 | Both coleorhiza and coleoptile are clearly visible, but the coleoptile is shorter |
| | than the length of the seed. No root elongation has occurred, although lateral |
| | adventitious root initials may be evident in some species. Plumule (coleoptile |
| | and first true leaf) has not begun to green. |
| 1.5 | Coleoptile is still noticeably shorter than the length of the seed, but root(s) are |
| | clearly visible and have begun to elongate. First true leaf has not yet emerged |
| | from the coleoptile. Plumule (coleoptile and first true leaf) may or may not be |
| | green, depending on the species. |
| 2.0 | Coleoptile is equal in length to the seed or at least not obviously different in |
| | length than the seed. First true leaf has not_yet emerged from the coleoptile. |
| | Plumule (coleoptile and first true leaf) may or may not be green, depending |
| | on the species. Roots are present and elongated (may be stunted). |
| 2.5 | Coleoptile is noticeably longer than length of the seed, but the first true leaf |
| | has not_yet emerged from the coleoptile. Plumule (coleoptile and first true |
| | leaf) may be green. Roots are present and elongated (may be stunted). |
| 3.0 | First true leaf has emerged from the coleoptile and is green in color. The |
| | emerged portion of the leaf is obviously shorter in length than the coleoptile. |
| | Roots are present and elongated. |
| 3.5 | First true leaf is fully emerged from the coleoptile and is green in color. The |
| | emerged_portion of the leaf is approximately equal in length to the coleoptile |
| | or at least not obviously shorter or longer than the length of the coleoptile. |
| | Roots are present and elongated. |
| 4.0 | First true leaf is fully emerged from the coleoptile and green in color. The |
| | emerged portion of the leaf is obviously longer than the length of the |
| | coleoptile. Roots are present and elongated. |

Assays of Culture Filtrates, Live Bacteria, and FVG-Analogs for Antibiosis Activity against Erwinia amylovora and Other Bacterial Species and Strains: The potential antibiosis activities of various P. fluorescens isolates, their culture filtrates, and the FVG analogs prepared as described above were tested against Erwinia amylovora and a number of other bacterial species and strains listed in Table 4. For this purpose, cryovial stocks of the test organisms (including P. fluorescens lines from the stocks described above) were prepared from bacterial cultures grown in LB liquid medium. For this specific experimental purpose, aliquots of these cultures were stored in cryovials at −80° C. in LB medium containing glycerol at a final concentration of 15% (v/v).

The LB Medium used here was taken from Sambrook, J. and Russell, D. W., Molecular Cloning: A Laboratory Manual, 3rd Edition. Cold Spring Harbor Laboratory Press, Cold Spring Harbor, N.Y., 2001). It contained 10 g Difco® Bacto-Tryptone, 5 g Difco Yeast Extract, and 10 g NaCl per liter of medium and was adjusted to pH 7.4 with NaOH. For semi-solid LB Medium (for stock plates), 15 g of Difco® Bacto-Agar were added to a liter of the liquid medium.

Bacterial lawns of test organisms were prepared from cells grown by inoculating 10 μL of the 15% glycerol stocks into 5 mL of liquid LB Medium. The cells were allowed to multiply overnight at 28° C. with shaking (200 rpm). The resulting bacterial suspension was adjusted with water to an optical density of 0.2 at 600 nm (Spectronic® 20 Spectrophotometer; Milton Roy Co., Rochester N.Y.). 300 μL aliquots of the diluted suspension were spread onto the agar surfaces of Petri plates (100×15 mm) containing mL/plate of 925 Minimal Medium (prepared and amended as described below). The surfaces of the plates were then dried briefly by removal of the lids in a sterile transfer hood, and the antibiosis assays described below were initiated immediately.

The 925 Minimal Medium used for preparation of the test lawns of bacteria was adapted from Langley and Kado (Mutation Research, 14: 277-286, 1972). Three stock solutions were used in media preparation: Stock A, containing 393 g K₂HPO₄.3H₂O (or 330 g anhydrous K₂HPO₄) and 100 g anhydrous NaH₂PO₄ per liter; Stock B, containing 100 g NH₄Cl and 39 g MgSO₄.7H₂O per liter; and Stock C (100× Vitamin Stock), containing 0.337 g Thiamine-HCl and 0.123 g Nicotinic Acid per 100 mL. Stock Solutions A and B were autoclaved and maintained in sterile condition at room temperature. Stock Solution C was filter-sterilized and stored at 4° C. To prepare the medium, 10 mL of Stock A and 10 mL of Stock B were added to about 500 mL of deionized water in a 1-liter volumetric flask, and the resulting solution was made to a final volume of 1 liter. The pH of this solution was adjusted to 6.8, 15 g of Difco®Bacto-Agar were added, followed by autoclave sterilization for 30 minutes. The resulting sterile agar solution was cooled for 20 minutes at 50° C. To this solution was added 1 mL of freshly prepared and filter-sterilized 0.1 M FeCl₃-H₂O, 20 mL of freshly prepared and filter-sterilized 10% (w/v) glucose solution, and 10 mL of Stock Solution C (100× Vitamin Stock). All additions were made in a transfer hood to maintain sterility. The final medium was dispensed to sterile plastic Petri plates (100×15 mm plates, 25 mL per plate).

For tests of culture filtrates and solutions of analogs for antibiosis activity, immediately after spreading lawns of the test organism, central wells were punched in the agar of the bacterial lawns using a sterile #6 cork borer. 300 μL of test solution were dispensed into each well. The plates were incubated at 28° C. for 48 hours and then examined for evidence of antibiosis activity.

For tests of live bacteria as potential biocontrol agents, the bacteria of interest were first grown as lawns or slants on LB Medium supplemented with 1.5% (w/v) Difco® Bacto-Agar. These cultures were incubated for 16 hours at 28° C. and then held at 4° C. until used. For assay of biocontrol activity, a sample of the bacteria was scooped from an agar stock plate with the tip of a sterile toothpick and a colony spotted at a marked location on a dried lawn of the bacterial species being tested for susceptibility to the biocontrol agent. After application of the test samples, the plates were incubated at 28° C. for 48 hours and then examined for evidence of antibiosis activity.

Thin-Layer Chromatographic Analysis (TLC Analysis) of Pseudomonas Culture Filtrates Measured volumes of bacterial culture filtrates were taken to dryness in vacuo at ≦45° C. Typically, 36 mL aliquots of culture filtrate were taken to dryness in 250-mL evaporation flasks. The dry solids remaining after evaporation of the filtrate were extracted three times (5 minutes per extraction) with 90% (v/v) ethanol. Each of these three extractions was performed by swirling the solids with a volume of solvent equal to one-third of the original volume of culture filtrate (e.g., 12 mL per extraction for samples originating from 36 mL of culture filtrate). The three extracts prepared in this manner were combined, taken to dryness in vacuo at ≦45° C., and the recovered solids immediately dissolved in a volume of 76% ethanol equivalent to 1/20 of the original volume of culture filtrate (e.g., 1.8 mL for an original culture filtrate volume of 36 mL). The resulting 76% ethanol solutions (20× concentration) were analyzed by chromatography on cellulose and silica gel TLC plates as described below.

Analtech® Microcrystalline Cellulose Analytical TLC plates (5×20 cm, 250μ thick layer) and Analtech® Analytical Silica Gel GHL TLC plates (5×20 cm, 250μ thick layer) were used for TLC analysis. Aliquots (200 μL) of the 76% ethanol solutions prepared by extracting dried culture filtrates as described above were streaked across each plate by repeated applications to a line 3 cm from one end of the plate. The 200 μL sample was applied with a 50 μL Lang-Levy micropipette equipped with a screw-type micropipette control device and using a filtered air-stream to dry the origin for 20 to 30 seconds between applications. The chromatograms were allowed to dry for 10 minutes following the final application and then developed over a distance of 12 cm in a cylindrical tank (6×22 cm) containing approximately 25 mL of one of the following solvents. For cellulose TLC plates, the developing solvent consisted of water:isopropanol:ethyl acetate (15:10:7.5). For silica gel TLC plates, the solvent consisted of ethyl acetate:isopropanol:methanol:water (5:5:18:2). The developed chromatograms were dried and sprayed with a ninhydrin reagent consisting of 0.25 grams ninhydrin in 95% ethanol containing 3.0 mL of glacial acetic acid per 100 mL of final solution. Chromatograms sprayed with this reagent were heated in an oven (80°-90° C.) for 15 minutes to develop the ninhydrin color.

NMR Spectroscopy: Samples of the culture filtrate extracts prepared for TLC analysis were taken for ¹H NMR data acquisition. For this purpose, 1.4 mL aliquots of the 20-fold concentrated 76% ethanol solutions, prepared as described above, were concentrated in vacuo at 37° C. and placed under high vacuum for up to 24 hr to remove all traces of protonated solvent. The resulting dry samples were resuspended in 20 microliters of deuterated water (D₂O). These extremely concentrated solutions were filtered through a 2 micron Upchurch Scientific Mini Microfilter before 5-10 microliters of the resulting filtrate were manually injected through a second inline microfilter into a Protasis microflow capillary NMR probe (2.5 microliter active coil volume; 5 microliter flow cell) installed in a Bruker Avance DRX300 NMR spectrometer. ¹H NMR spectra were acquired in 256 scans at 298K on each sample and internally referenced to the residual HOD solvent peak calibrated to δ_{H} 4.80 ppm.

### B. Results

Effects of Pseudomonas fluorescens WH6 and WH6 Culture Filtrate on Erwinia amylovora

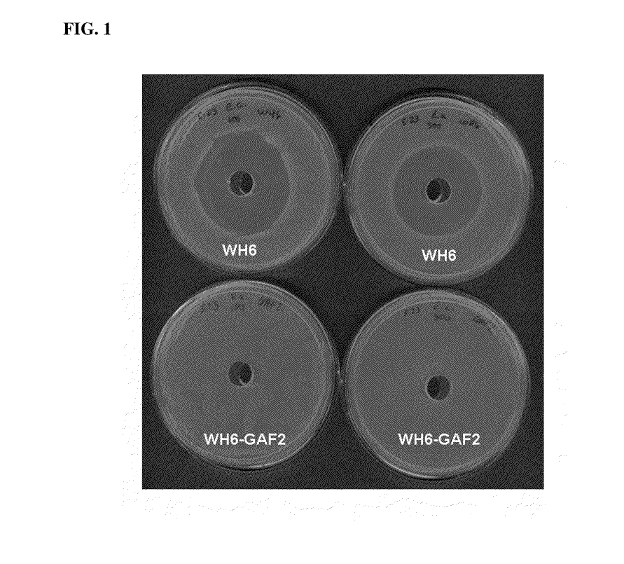

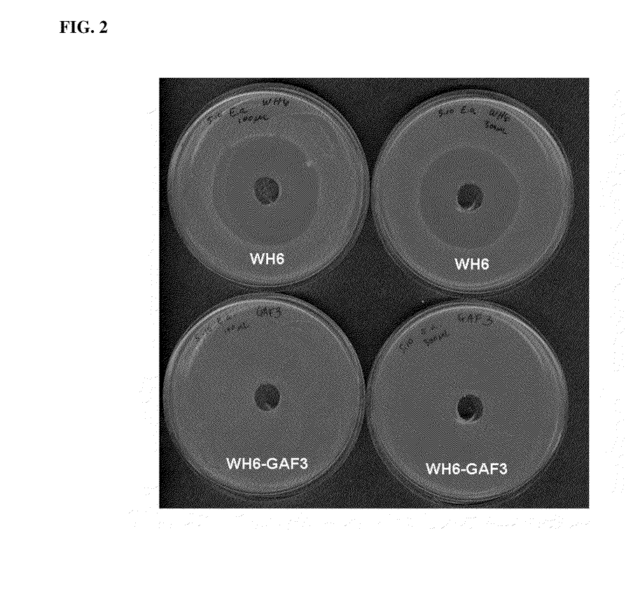

The inhibitory effects of FVG on Erwinia amylovora were demonstrated by comparing the effects of culture filtrate from the FVG-producing organism Pseudomonas fluorescens WH6 with the effects of culture filtrates from mutants of WH6 that had been shown previously to have lost the ability to produce FVG. (The characteristics of P. fluorescens WH6 and the GAF2 and GAF3 mutants of WH6 are described in U.S. Patent Publication 2006/0147438). To test the effects of the culture filtrates on E. amylovora, Petri dishes containing an appropriate agar medium were spread with E. amylovora inocula as described above. A sterile cork borer was then used to punch a central well in the freshly inoculated agar medium, and an appropriate volume of test solution was transferred to the well. The results of a comparison of WH6 culture filtrate and culture filtrate from the GAF2 mutant of WH6 are shown in FIG. 1. As can be seen in this figure, a zone of inhibition surrounds the wells containing WH6 culture filtrate, but no such zone is evident around the wells containing culture filtrate from the GAF2 mutant previously shown to have lost the ability to synthesize FVG. Similar results were obtained in tests using culture filtrate from the GAF3 mutant (as shown in FIG. 2), which was also previously shown to have lost the ability to produce FVG. Thus, the inhibitory effect of WH6 culture filtrate on the growth of E. amylovora can be attributed to the FVG content of the filtrate.

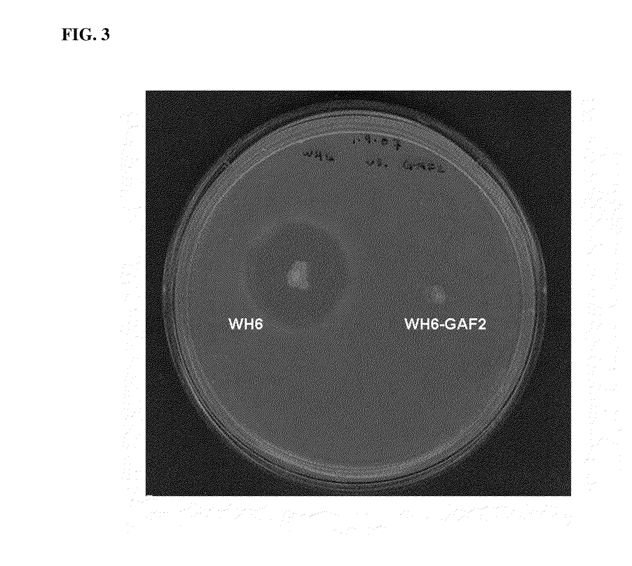

P. fluorescens WH6 bacteria are able to inhibit E. amylovora directly, as shown in FIG. 3. In this case, a colony of WH6 bacteria was inoculated at the indicated site in a freshly spread lawn of E. amylovora. After 48 hours incubation, a zone of inhibition was evident around the WH6 colony, whereas no such inhibition was visible surrounding a colony of the GAF2 mutant introduced on the same plate at the same time.

Effects on E. amylovora of Culture Filtrates and Bacterial Colonies from the Other FVG-Producing Pseudomonas Isolates Described in US Patent Publication 2006/0147438

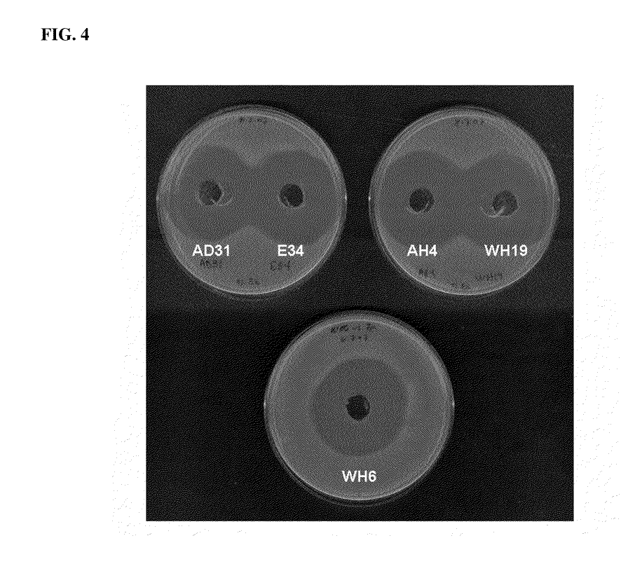

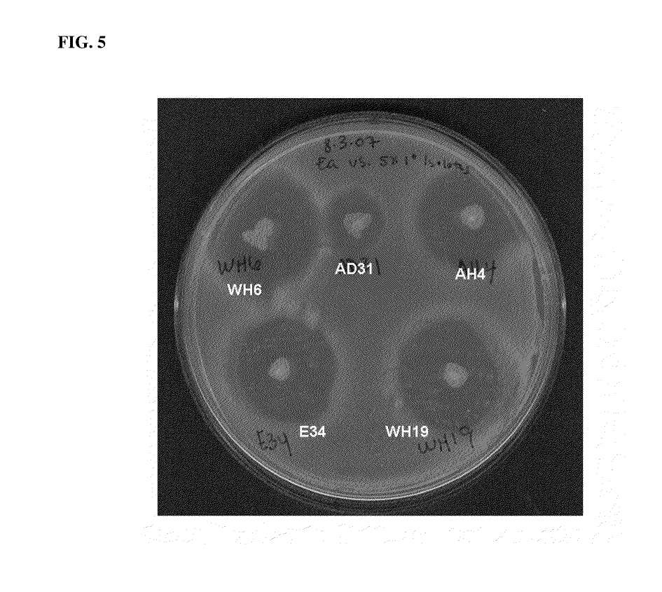

The referenced patent application described, in addition to WH6, four other Pseudomonas isolates that produced the compound then designated as a Germination-Arrest Factor (GAF) and subsequently identified as FVG as described in U.S. Patent application Ser. No. 12/567,590. The effects on E. amylovora of culture filtrates from all five of these isolates are compared in FIG. 4. As expected, all five FVG-containing culture filtrates produced zones of inhibition in the E. amylovora lawns. Similar results were obtained when live bacteria from the five isolates were inoculated onto Erwinia lawns (FIG. 5). These results are also consistent with, and support the claim of, an inhibitory action of FVG on the growth of E. amylovora.

Effects on E. amylovora of Additional Pseudomonas Isolates and Culture Filtrates Characterized Since Jun. 1, 2005

Since the filing of the referenced patent application, a number of additional isolates of Pseudomonas fluorescens and other bacterial species have been tested for antibiotic activity. The culture filtrates from these isolates and strains have been tested for GAF activity, for ability to inhibit E. amylovora, and for the presence of FVG. The specific sources of these bacterial cell lines and the criteria used for their taxonomic identification are shown in Tables 2 and 3. All of the isolates listed here were obtained from soils of the Willamette Valley and were selected on the basis of initial tests that indicated that the live bacteria had some herbicidal activity against grassy weeds. The laboratory strains of P. fluorescens tested here were selected on the basis of availability. The genomes of two of these laboratory strains have been sequenced (Pf-5, genome available at GenBank Accession No. NC_{—}004129, and PfO-1, genome available at GenBank Accession No. CP000094).

Culture filtrates from seventeen of twenty P. fluorescens isolates and strains tested in our standard Poa bioassay were found to possess GAF activity (Table 4), and three of eight bacterial isolates identified as belonging to other species also yielded culture filtrates that exhibited GAF activity in this bioassay (Table 5). None of the three laboratory strains of P. fluorescens tested here was found to produce GAF activity (Table 4).

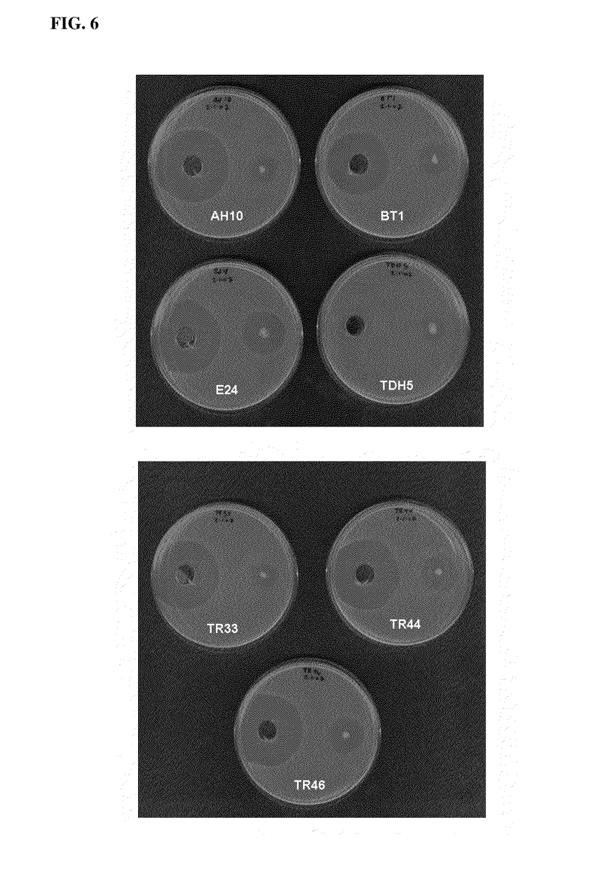

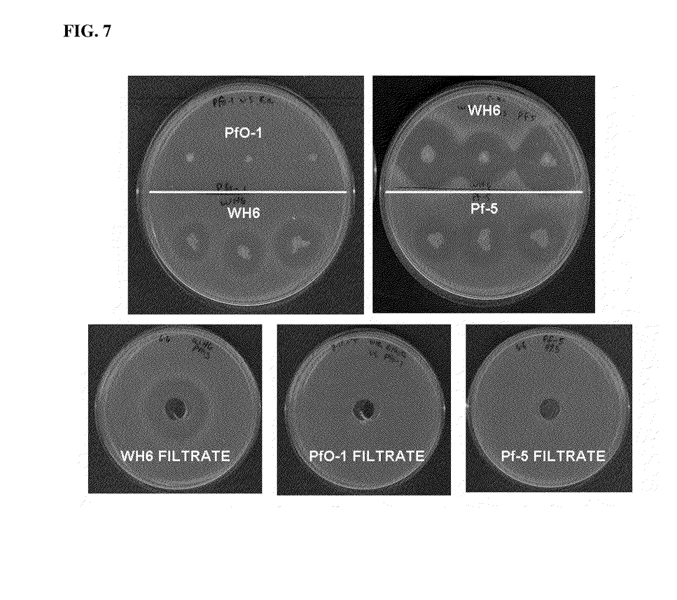

Culture filtrates from all of the isolates and strains listed in Tables 2 and 3 were tested for anti-Erwinia activity as described above. As shown in Table 6, all of the culture filtrates that were found to possess GAF activity also exhibited anti-Erwinia activity in our Petri plate assays. Representative results for seven of the P. fluorescens isolates are illustrated in FIG. 6. Both the live colonies and culture filtrates from the six GAF-producing isolates shown here inhibited the growth of E. amylovora on the test plates, while TDH5, an isolate that was inactive in the Poa bioassay, was slightly active in both tests for anti-Erwinia activity. This low level of activity is clearly by a means other than via FVG, as TDH5 is inactive in the Poa bioassay and lacks the GAF-specific ninhydrin band. Comparisons of the anti-Erwinia activity of P. fluorescens WH6 and two of the two P. fluorescens strains obtained from other laboratories (Pf-5 and PfO-1) are illustrated in FIG. 7. As expected from the fact that neither strain produces GAF (FVG), culture filtrates prepared from these strains were inactive in inhibiting the growth of E. amylovora. Similarly, live colonies of PfO-1 were inactive in the Erwinia assay. However, live colonies of Pf-5 did give zones of inhibition in E. amylovora lawns, suggesting that the production of an antimicrobial compound by Pf-5 is induced under these growth conditions.

TABLE 2
Origin and Taxonomic Identification of Additional Characterized
Pseudomonas fluorescens Isolates. Isolates with GAF activity that have been shown
to produce FVG are marked with an asterisk.

| | SITE OF | | | |
|---|---|---|---|---|
| | ISOLATION | | | |
| | or | RHIZOSPHERE | TAXONOMIC IDENTIFICATION | |
| | LABORATORY | SOURCE | FAME | rDNA |
| ISOLATE | SOURCE | OF ISOLATE | ANALYSIS | SEQUENCING |
| A17 | Disturbed Site, Alsea | Poa Species | Pseudomonas | Pseudomonas |
| | Valley | | fluorescens | fluorescens |
| | Benton County, OR | | Biotype B | |
| A3422A* | Disturbed Site, Alsea | Unknown | Pseudomonas | Pseudomonas |
| | Valley | | fluorescens | fluorescens |
| | Benton County, OR | | Biotype G | |
| AH7 | Alsea Valley | Poa Species | Pseudomonas | Pseudomonas |
| | Benton county, OR | | fluorescens | fluorescens |
| | | | or P. putida Biotype B | |
| AH10* | Disturbed Site, Alsea | Poa Species | Pseudomonas | Pseudomonas |
| | Valley | | fluorescens | fluorescens |
| | Benton County, OR | | Biotype G/taetrolens | |
| ALW38* | Lawn, Alsea Valley | Poa Species | Pseudomonas | Pseudomonas |
| | Benton County, OR | | fluorescens | fluorescens |
| | | | Biotype G | |
| BT1* | Botany Research | Wheat Cultivar | Pseudomonas | Pseudomonas |
| | Farm, OSU | | fluorescens | fluorescens |
| | Linn County, OR | | Biotype A | |
| E24* | Hyslop Research | Poa Species | Pseudomonas | Pseudomonas |
| | Farm, OSU | | fluorescens | fluorescens |
| | Benton County, OR | | Biotype B | |
| G2Y* | Grower's Field | Lolium perenne | Pseudomonas putida | Pseudomonas |
| | Linn County, OR | | Biotype B | fluorescens |
| GTR12* | Edge of Compost | Grassy Weeds | Pseudomonas | Pseudomonas |
| | Pile, Farm | | fluorescens | fluorescens |
| | Philomath, Benton | | Biotype B | |
| | County, OR | | | |
| GTR24* | Edge of Compost | Grassy Weeds | Pseudomonas | Pseudomonas |
| | Pile, Farm | | fluorescens | fluorescens |
| | Philomath, Benton | | Biotype B | |
| | County, OR | | | |
| GTR40* | Philomath, Benton | Grassy Weeds | Pseudomonas | Pseudomonas |
| | County, OR | | fluorescens | fluorescens |
| | Edge of Compost | | Biotype B | |
| | Pile, Farm | | | |
| | Philomath, Benton | | | |
| | County, OR | | | |
| HB14* | Lawn, Alsea Valley | Poa Species | Pseudomonas putida | Pseudomonas |
| | Benton County, OR | | Biotype B | fluorescens |
| HB26* | Lawn, Alsea Valley | Poa Species | Pseudomonas putida | Pseudomonas |
| | Benton County, OR | | Biotype B | fluorescens |
| HB32* | Lawn, Alsea Valley | Poa Species | Pseudomonas putida | Pseudomonas |
| | Benton County, OR | | Biotype B | fluorescens |
| ST22* | Hyslop Research | Hordeum vulgare | Pseudomonas | Pseudomonas |
| | Farm, OSU | | fluorescens | fluorescens |
| | Benton County, OR | | Biotype G | |
| TDH5 | Private Vegetable | Poa Species | Pseudomonas | Pseudomonas |
| | Farm | | fluorescens | fluorescens |
| | Benton County, OR | | Biotype B | |
| TR33* | Botany Research | Triticale Species | Pseudomonas | Pseudomonas |
| | Farm, OSU | | fluorescens | fluorescens |
| | Linn County, OR | | Biotype A | |
| TR44* | Botany Research | Triticale Species | Pseudomonas | Pseudomonas |
| | Farm, OSU | | fluorescens | fluorescens |
| | Linn County, OR | | Biotype A | |
| TR46* | Botany Research | Triticale Species | Pseudomonas | Pseudomonas |
| | Farm, OSU | | fluorescens | fluorescens |
| | Linn County, OR | | Biotype G/taetrolens | |
| W36* | Hyslop Research | Triticum | Pseudomonas | Pseudomonas |
| | Farm, OSU | (with Poa species) | fluorescens | fluorescens |
| | Benton County, OR | | Biotype G | |
| A506 | Dr. Joyce Loper | Leaf surface | Pseudomonas putida | Pseudomonas |
| | USDA-ARS, | of Pyrus | Biotype A | fluorescens |
| | Corvallis, OR | | | |

| | | | |
|---|---|---|---|
| Pf-5 | Dr. Joyce Loper | Gossypium | Pseudomonas fluorescens |
| | USDA-ARS, | hirsutum | Identification Provided by Source |
| | Corvallis, OR | | |
| PfO-1 | Dr. Stewart Levy | Agricultural Loam | Pseudomonas fluorescens |
| | Tufts University | | Identification Provided by Source |

TABLE 3
Origin and Taxonomic Identification of Isolates of Additional
Characterized Bacterial Species (other than P. fluorescens). Isolates with GAF
activity that have been shown to produce FVG are marked with an asterisk.

| | SITE OF | | | |
|---|---|---|---|---|
| | ISOLATION | | | |
| | or | RHIZOSPHERE | TAXONOMIC IDENTIFICATION | |
| | LABORATORY | SOURCE | FAME | rDNA |
| ISOLATE | SOURCE | OF ISOLATE | ANALYSIS | SEQUENCING |
| A3203* | Willamette Valley, | Unknown | Enterobacter sp. | Enterobacter |
| | OR | | | kobei |
| A342* | Willamette Valley, | Unknown | Pseudomonas | Pseudomonas |
| | OR | | fluorescens | mucidolens |
| | | | Biotype G | or P. synxantha |
| AH18 | Lawn, Alsea Valley | Poa Species | Pseudomonas | Pseudomonas |
| | Benton county, OR | | fluorescens | mucidolens |
| | | | Biotype B | or P. synxantha |
| BW1 | Hyslop Research | Triticum | Pseudomonas | Pseudomonas |
| | Farm, OSU | (with Poa species) | fluorescens | poae |
| | Benton County, OR | | Biotype B | or P. trivialis |
| GTR20- | Edge of Compost | Grassy Weeds | Enterobacter | Enterobacter |
| 18-2 | Pile, Farm | | intermedius | amnigenus |
| | Philomath, Benton | | | |
| | County, OR | | | |
| GTR28 | Edge of Compost | Grassy Weeds | Pseudomonas putida | Pseudomonas |
| | Pile, Farm | | Biotype A | veronii |
| | Philomath, Benton | | | |
| | County, OR | | | |
| L2-1-1 | Organic Vegetable | Poa species | Enterobacter | Enterobacter |
| | Farm | | intermedius | asburiae |
| | Philomath, Benton | | | |
| | County, OR | | | |
| TDH40* | Organic Vegetable | Poa species | Pseudomonas | Pseudomonas |
| | Farm | | fluorescens | mucidolens |
| | Philomath, Benton | | Biotype B | or P. synxantha |
| | County, OR | | | |

TABLE 4
Germination-Arrest Activity of Culture Filtrates from Additional
Pseudomonas fluorescens Isolates and Strains Tested in the Standard
Poa Bioassay. Isolates with GAF activity that have been shown to
produce FVG are marked with an asterisk.

| | MEAN GERMINATION SCORE | | |
|---|---|---|---|
| | IN THE POA BIOASSAY AT THE | | |
| | INDICATED CULTURE FILTRATE DILUTION | | |
| | (±Standard Error of the Mean) | | |
| Isolate | 0.1X | 0.3X | 1.0X (Full-Strength) |
| PMS | 4.0 ± 0.00 | 4.0 ± 0.00 | 4.0 ± 0.00 |
| Medium | | | |
| WH6* | 1.0 ± 0.00 | 1.0 ± 0.00 | 1.0 ± 0.00 |
| A17 | 4.0 ± 0.00 | 4.0 ± 0.00 | 3.5 ± 0.09 |
| A3422A* | 1.0 ± 0.00 | 1.0 ± 0.00 | 1.0 ± 0.00 |
| AH7 | 4.0 ± 0.00 | 4.0 ± 0.00 | 3.0 ± 0.17 |
| AH10* | 1.0 ± 0.00 | 1.0 ± 0.00 | 1.0 ± 0.00 |
| ALW38* | 1.4 ± 0.06 | 1.0 ± 0.00 | 1.0 ± 0.00 |
| BT1* | 1.7 ± 0.12 | 1.5 ± 0.0 | 1.0 ± 0.00 |
| E24* | 1.0 ± 0.0 | 1.0 ± 0.0 | 1.0 ± 0.0 |
| G2Y* | 1.9 ± 0.21 | 1.0 ± 0.00 | 1.0 ± 0.00 |
| GTR12* | 3.3 ± 0.26 | 1.0 ± 0.00 | 1.0 ± 0.00 |
| GTR24* | 1.2 ± 0.13 | 1.0 ± 0.00 | 1.0 ± 0.00 |
| GTR40* | 1.1 ± 0.08 | 1.0 ± 0.00 | 1.0 ± 0.00 |
| HB14* | 1.0 ± 0.00 | 1.0 ± 0.00 | 1.0 ± 0.00 |
| HB26* | 1.4 ± 0.07 | 1.0 ± 0.00 | 1.0 ± 0.00 |
| HB32* | 1.2 ± 0.13 | 1.0 ± 0.00 | 1.0 ± 0.00 |
| ST22* | 1.8 ± 0.20 | 1.0 ± 0.00 | 1.0 ± 0.00 |
| TDH5 | 4.0 ± 0.00 | 4.0 ± 0.00 | 2.8 ± 0.15 |
| TR33* | 1.0 ± 0.00 | 1.0 ± 0.00 | 1.0 ± 0.00 |
| TR44* | 1.0 ± 0.00 | 1.0 ± 0.00 | 1.0 ± 0.00 |
| TR46* | 1.0 ± 0.00 | 1.0 ± 0.00 | 1.0 ± 0.00 |
| W36* | 1.0 ± 0.00 | 1.0 ± 0.00 | 1.0 ± 0.00 |
| A506 | 4.00 ± 0.00 | 4.0 ± 0.00 | 3.9 ± 0.08 |
| Pf5 | 4.0 ± 0.00 | 4.0 ± 0.00 | 4.0 ± 0.00 |
| PfO-1 | 4.0 ± 0.00 | 4.0 ± 0.00 | 3.4 ± 0.13 |

PMS Medium and WH6* are controls

TABLE 5
Germination-Arrest Activity of Culture Filtrates from Additional
Bacterial Species (Other than P. fluorescens) Tested in the Standard
Poa Bioassay. Isolates with GAF activity that have been shown to
produce FVG are marked with an asterisk.

| | | MEAN GERMINATION SCORE | | |
|---|---|---|---|---|
| | | IN THE POA BIOASSAY | | |
| | | AT THE INDICATED CULTURE | | |
| SOURCE OF CULTURE | | FILTRATE DILUTION | | |
| FILTRATE | | (±Standard Error | | |
| | SPECIES | of the Mean) | | |
| | (from rDNA | | | 1.0X |
| ISOLATE | Sequencing) | 0.1X | 0.3X | (full-strength) |
| PMS | — | 4.0 ± 0.00 | 4.0 ± 0.00 | 4.0 ± 0.00 |
| Medium | | | | |
| WH6* | P. fluorescens | 1.0 ± 0.00 | 1.0 ± 0.00 | 1.0 ± 0.00 |
| A3203* | Enterobacter kobei | 1.2 ± 0.00 | 1.0 ± 0.00 | 1.0 ± 0.00 |
| A342* | P. mucidolens | 1.5 ± 0.00 | 1.0 ± 0.00 | 1.0 ± 0.00 |
| | or P. synxantha | | | |
| AH18 | P. mucidolens | 4.0 ± 0.00 | 4.0 ± 0.00 | 2.5 ± 0.17 |
| | or P. synxantha | | | |
| BW1 | P. poaeor | 4.0 ± 0.00 | 4.0 ± 0.00 | 4.0 ± 0.00 |
| | P. trivialis | | | |
| GTR20- | Enterobacter | 4.0 ± 0.00 | 4.0 ± 0.00 | 4.0 ± 0.00 |
| 18-2 | amnigenus | | | |
| GTR28 | P. veronii | 4.0 ± 0.00 | 4.0 ± 0.00 | 4.0 ± 0.00 |
| L2-1-1 | Enterobacter | 4.0 ± 0.00 | 4.0 ± 0.00 | 4.0 ± 0.00 |
| | asburiae | | | |
| TDH40* | P. mucidolens | 1.5 ± 0.00 | 1.0 ± 0.00 | 1.0 ± 0.00 |
| | or P. synxantha | | | |

PMS Medium and WH6* are controls

TABLE 6
Anti-Erwinia Activity of Culture Filtrates from Various Pseudomonas and
Enterobacter Isolates and Strains Tested in the Standard Erwinia
Bioassay. Filtrate from each isolate was tested in triplicate. Isolates
with GAF activity that have been shown to produce FVG are marked
with an asterisk.

| CULTURE | |
|---|---|
| FILTRATES | |
| from | |
| Pseudomonas | ZONE OF INHIBITION |
| fluorescens | (Area in CM² ± |
| ISOLATES | Standard Error of the Mean) |
| A17 | 0.0 ± 0.00 |
| A3422A* | 10.6 ± 0.08 |
| AH7 | 0.0 ± 0.00 |
| AH10* | 9.2 ± 0.30 |
| ALW38* | 14.4 ± 0.23 |
| BT1* | 6.1 ± 0.19 |
| E24* | 7.4 ± 0.19 |
| G2Y* | 12.9 ± 0.23 |
| GTR12* | 12.2 ± 0.15 |
| GTR24* | 10.9 ± 0.62 |
| GTR40* | 9.6 ± 0.03 |
| HB14* | 12.4 ± 0.49 |
| HB26* | 9.9 ± 0.21 |
| HB32* | 9.9 ± 0.08 |
| ST22* | 9.0 ± 0.10 |
| TDH5 | 0.0 ± 0.00 |
| TR33* | 8.3 ± 0.14 |
| TR44* | 9.2 ± 0.16 |
| TR46* | 11.2 ± 0.23 |
| W36* | 10.0 ± 0.21 |
| A506 | 0.0 ± 0.00 |
| Pf-5 | 0.0 ± 0.00 |
| PfO-1 | 0.0 ± 0.00 |

| CULTURE FILTRATES | | |
|---|---|---|
| From | | |
| | SPECIES | ZONE OF INHIBITION |
| | (from rDNA | (Area in CM² ± |
| ISOLATE | Sequencing) | Standard Error of the Mean) |
| A3203* | Enterobacter kobei | 10.8 ± 0.39 |
| A342* | Pseudomonas | 7.0 ± 0.33 |
| | mucidolens | |
| | or P. synxantha | |
| AH18 | Pseudomonas | 0.0 ± 0.00 |
| | mucidolens | |
| | or P. synxantha | |
| BW1 | Pseudomonas poae | 0.8 ± 0.05 |
| | or P. trivialis | |
| GTR20-18-2 | Enterobacter | 2.6 ± 0.61 |
| | amnigenus | |
| GTR28 | Pseudomonas veronii | 0.5 ± 0.21 |
| L2-1-1 | Enterobacter | 2.2 ± 0.36 |
| | asburiae | |
| TDH40* | Pseudomonas | 7.7 ± 0.31 |
| | mucidolens | |
| | or P. synxantha | |

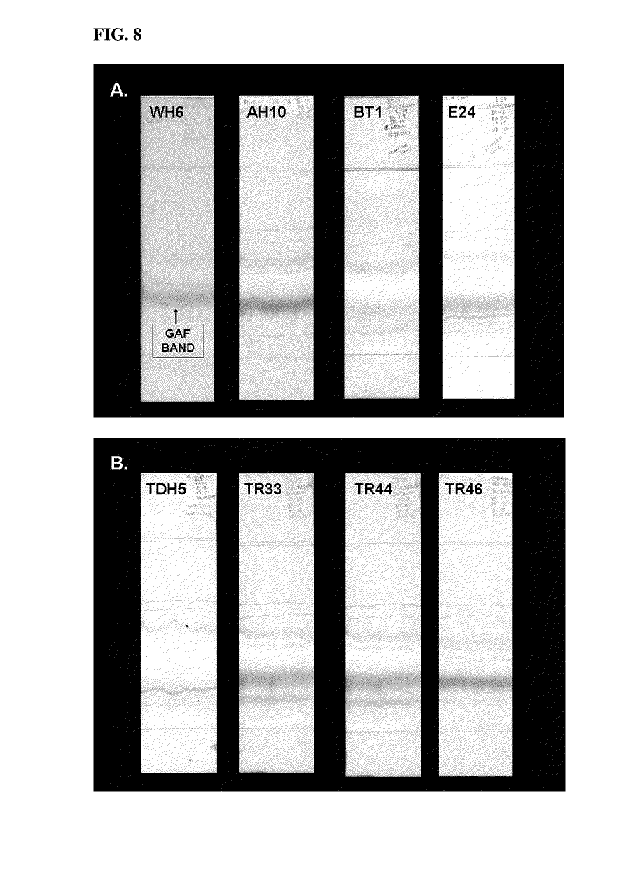

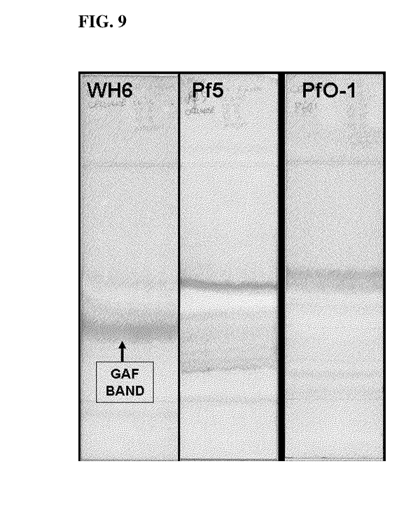

All of the culture filtrates screened for GAF activity and for anti-Erwinia activity were subsequently analyzed for FVG by thin-layer chromatography (TLC) and by nuclear magnetic resonance (NMR) spectroscopy. TLC analysis of culture filtrates prepared from the isolates and strains listed in Tables 2 and 3 demonstrated that all of the culture filtrates that exhibited GAF activity and anti-Erwinia activity contained the expected ninhydrin-reactive band characteristic of FVG. This band was absent from the isolates and strains that were inactive in the Poa bioassay (e.g. isolates A17, AH7, TDH5, AH18, BW1, GTR20-18-2, and L2-1-1, as well as the laboratory strains A506, Pf-5, and PfO-1). Representative results of TLC analyses are shown in FIG. 8 and FIG. 9.

FIG. 8 shows the ninhydrin-staining pattern of TLC chromatograms prepared from samples of the culture filtrates of seven GAF-producing (i.e. FVG-producing) isolates of P. fluorescens (WH6, AH10, BT1, E24, TR33, TR44, and TR46) and one isolate (TDH5) that lacked GAF activity in the Poa assay. The chromatogram prepared from isolate BT1, an isolate which exhibited only weak GAF activity, had a correspondingly weak FVG band. In FIG. 9, the ninhydrin-staining pattern of the TLC chromatogram derived from culture filtrates of the FVG-producing bacterial strain, P. fluorescens WH6, is compared with that of two of the three laboratory strains that were inactive in both the Poa bioassay and the anti-Erwinia assay. As expected, culture filtrates from both strains (Pf-5 and PfO-1) were found by TLC analysis to lack the ninhydrin-reactive band characteristic of FVG. The results presented here are intended only as representative and illustrative. Similar TLC data were obtained for all of the isolates and strains listed in Tables 2 and 3. Thus, all of the strains and isolates that possessed GAF activity and anti-Erwinia activity yielded chromatograms that exhibited the characteristic FVG band. Conversely, this band was absent from chromatograms prepared from culture filtrates of the isolates and stains listed in Tables 2 and 3 that lacked such biological activity.

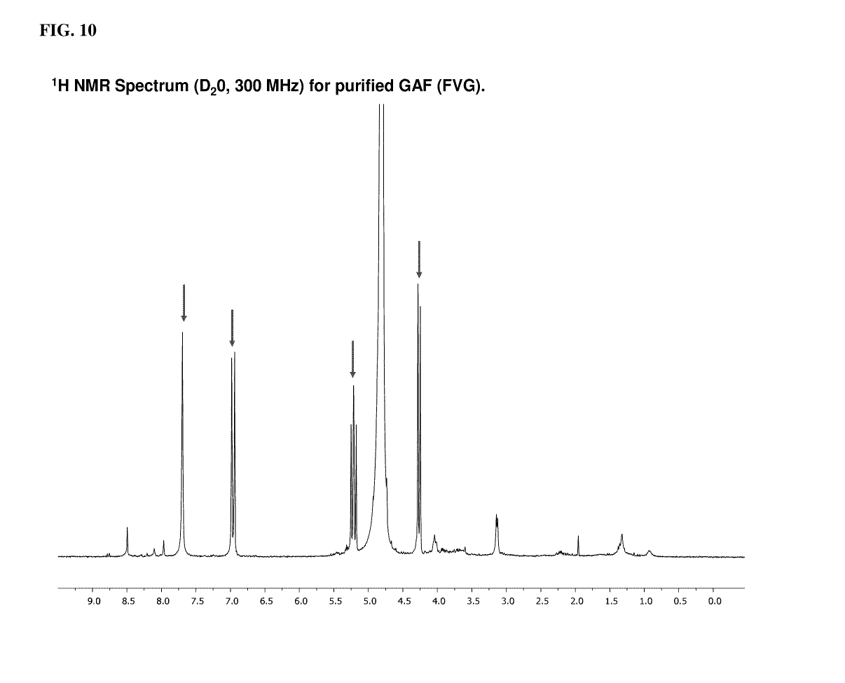

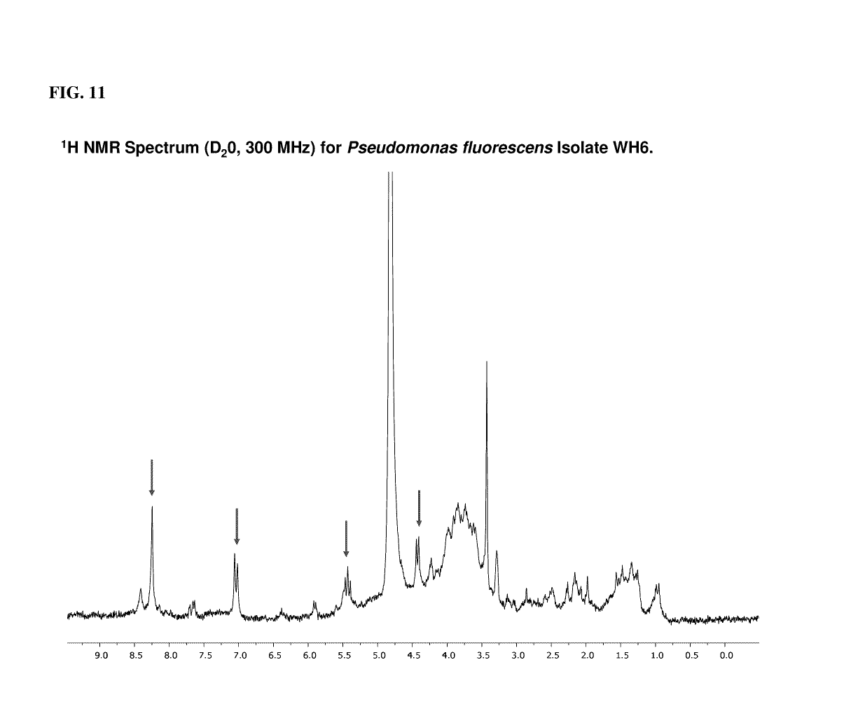

Nuclear magnetic resonance (NMR) spectroscopy of crude extracts prepared from culture filtrates of these bacterial isolates and strains confirmed the presence of FVG in all of the culture filtrates that exhibited GAF activity and anti-Erwinia activity. As illustrated in FIG. 10, the ¹H NMR spectrum of FVG in D₂O exhibits characteristic signals at δ_{H} 7.70, 4.26, 5.22, and 6.96 ppm corresponding respectively to the formyl group proton (hydrogen) of FVG, and the protons associated with the alpha (CH-2, beta (CH-3), and gamma (CH-4) positions in FVG. The latter three signals are characteristic of all oxyvinylglycines, while the former is specific for FVG itself. Four corresponding FVG proton signals are also clearly visible in the NMR spectrum of a crude 90% ethanol extract of dried culture filtrate from WH6 (FIG. 11). Note that in the spectra of the 90% ethanol extract of WH6 and other FVG-containing isolates, the four proton chemical shifts are deshielded (δ_{H} 8.19-8.25, 4.34-4.42, 5.37-5.43, and 6.98-7.03 ppm) relative to that in the spectrum for the purified FVG (δ_{H} 7.70, 4.26, 5.22, and 6.96 ppm). This is due to the known electronic effects (induced dipoles) of counter ions supplied by the different salt compositions in the crude and purified materials.

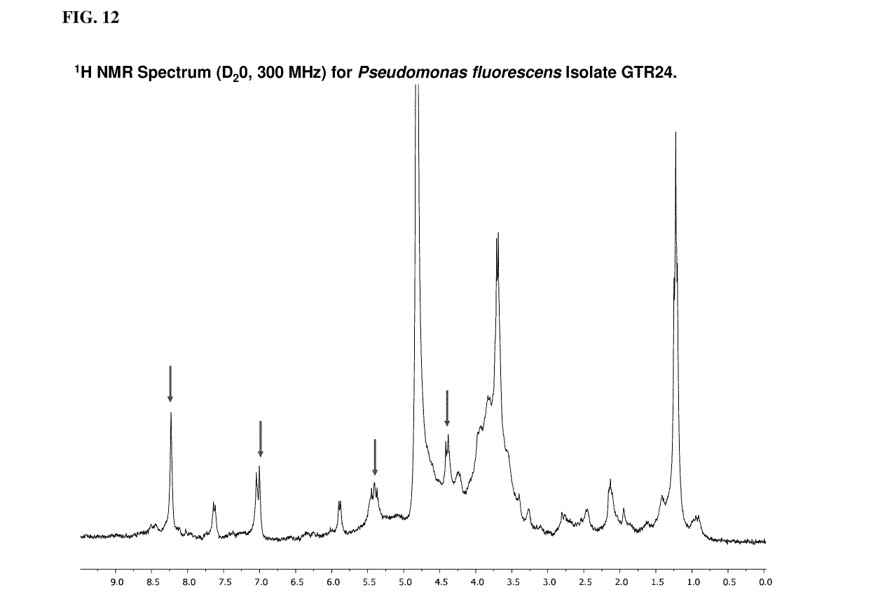

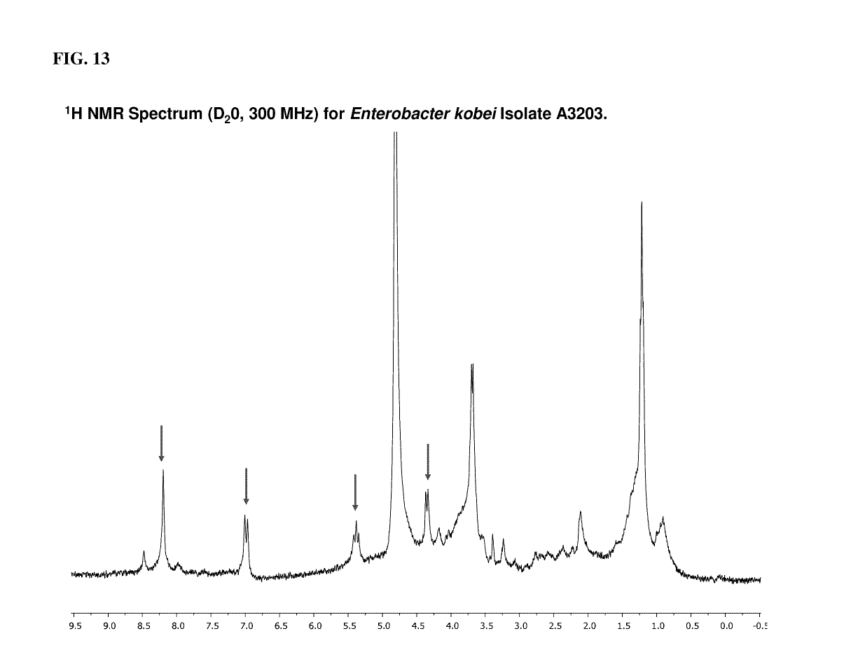

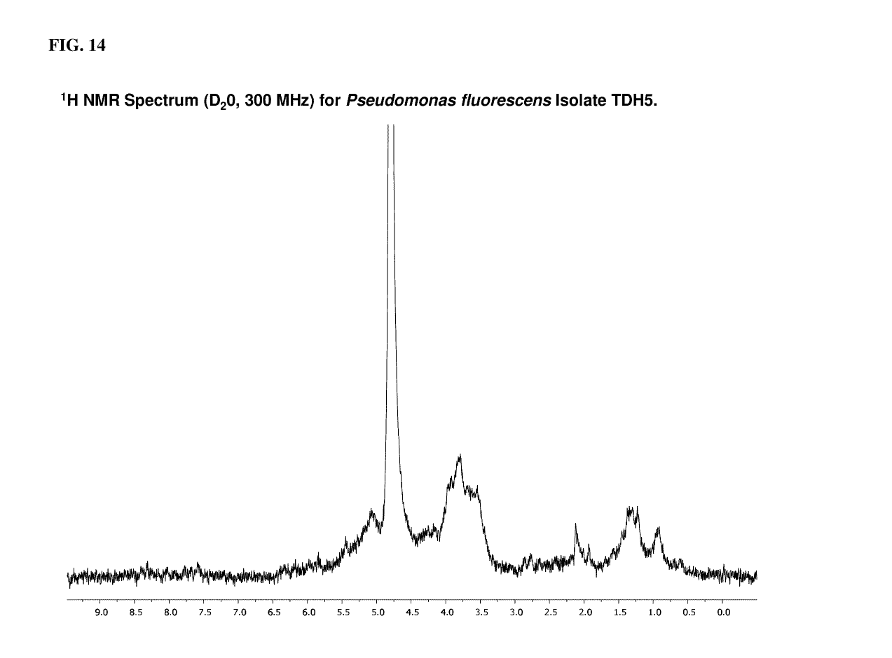

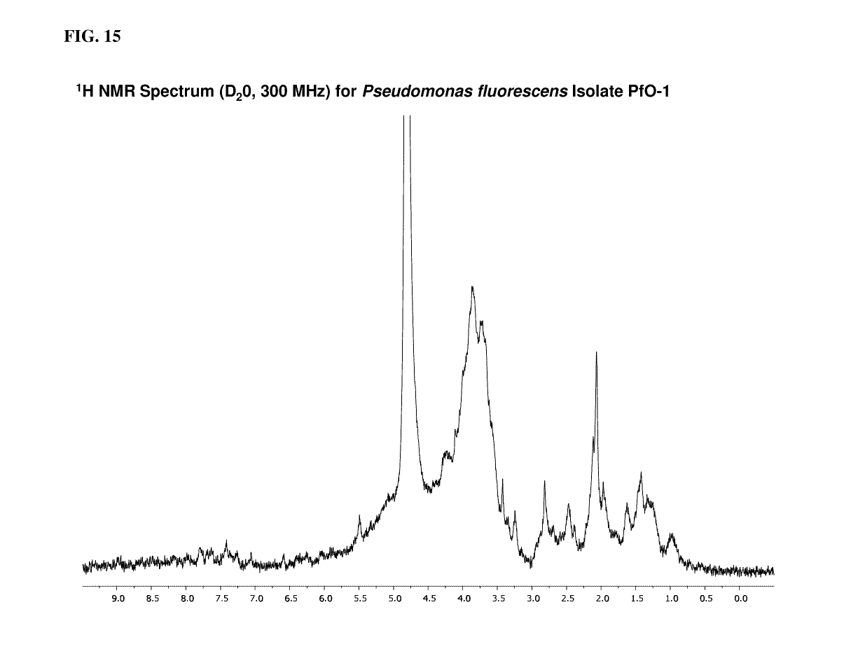

FIGS. 12 and 13 illustrate the NMR spectra obtained on crude extracts of the culture filtrates for two other FVG-producing isolates (Pseudomonas fluorescens Isolate GTR24 and Enterobacter kobei Isolate A3203). The arrows indicate the four distinctive NMR signals for FVG. In comparison, FIGS. 14 and 15 show the NMR spectra for two bacterial lines (P. fluorescens Isolate TDH5 and P. fluorescens Strain PfO-1) that lack the biological activity and TLC-banding pattern expected for FVG-producing bacteria. The four ¹H NMR signals characteristic of FVG are absent from these spectra. The analogous data were obtained for all of the bacterial isolates and strains listed in Tables 2 and 3. In all cases, the ¹H NMR signals characteristic of FVG could be detected in extracts of culture filtrates from bacterial lines that exhibited GAF activity and anti-Erwinia activity, while those signals were absent from the corresponding extracts prepared from culture filtrates of bacterial isolates and strains that lacked the indicated biological activities.

Effects of Pseudomonas fluorescens WH6 Culture Filtrate on Bacterial Species Other than Erwinia amylovora

The specificity of the antimicrobial activity of WH6 culture filtrate against E. amylovora was examined by 925-agar plate zone of inhibition tests, as described above. The results of these tests are summarized in Table 7.

TABLE 7
Effects of Pseudomonas fluorescens WH6 Culture Filtrate
on Various Bacterial Species

| | | Response to WH6 |
|---|---|---|
| Test Species | Strains/Pathovars | Culture Filtrate |
| Bacillus megaterium | Km | Strong |
| Erwinia amylovora | 153 | Inhibition |
| Erwinia carotovora | LA128 | Slight |
| Erwinia herbicola | Eh252, C9-1 | Inhibition |
| Escherichia coli | HB101 | |
| Agrobacterium radiobacter | K1026bv2 | No |
| Agrobacterium tumefaciens | C58bv1, B49c/83bv2, | Inhibition |
| | A348 | |
| Agrobacterium vitis | IL20bv3 | |
| Bradyrhizobium japonicum | USDA110 | |
| Pseudomonas fluorescens | PfO-1, Pf5, A506 | |
| Pseudomonas putida | NIR | |
| Pseudomonas syringae | phaseolicola, syringae, | |
| | tomato | |
| Rhodococcus fascians | A3b, A44a rif, 02-815, | |
| | A25 rif | |
| Xanthomonas campestris | ATCC33919, KXCC1 | |

As can be seen from these results, the antimicrobial activity of FVG appears to be restricted to a rather narrow range of organisms. Bacillus megaterium was the only bacterium tested that showed an inhibitory response similar to that of E. amylovora. Other species of Erwinia exhibited only small responses to the WH6 culture filtrate. None of the Pseudomonas species and strains tested was sensitive to the filtrate. Thus, the antimicrobial activity of FVG appears to be restricted to a rather narrow spectrum of microbes. This property of FVG may have important practical implications because it suggests that this compound and/or the organisms that produce it would have minimal environmental impact if utilized for the control of fire blight.

### Summary

The results reported in this example demonstrate that FVG-producing bacteria and culture filtrates from these organisms exert a pronounced inhibitory effect on the growth and multiplication of Erwinia amylovora, the causal agent of fire blight. Mutational analysis of the FVG-producing isolate Pseudomonas fluorescens WH6 has demonstrated that the active agent responsible for the anti-Erwinia activity of these organisms and filtrates is in fact FVG. Bacterial strains that do not produce FVG are either without effect on E. amylovora (e.g. P. fluorescens PfO-1) or inhibit E. amylovora through the production of compounds other than FVG. The antimicrobial effects of FVG have been shown to be restricted to a very narrow range of microbes.

### Example 2

### Natural and Synthetic Vinylglycines, and Natural Vinylglycine Analogs as Agents for the Control of Erwinia amylovora

This example shows that FVG structural analogs such as AVG (4-aminoethyoxyvinylglycine) are inhibitory to E. amylovora growth. Similarly, this example also shows that aminooxyacetic acid (AOA), a compound that, like vinylglycines, inhibits pyridoxal-phosphate-dependent enzymatic reactions, is also able to inhibit the growth of E. amylovora.

### A. Methods

Assays of E. amylovora growth inhibition were as described in Example 1. Aminoethoxyvinylglycine-hydrochloride (AVG, as ReTain®) and aminooxyacetic acid [AOA, O-(Carboxymethyl)hydroxylamine-hemihydrochloride, Sigma-Aldrich C13408-1G] were prepared as 30 mM solutions adjusted to pH 6.4 with KOH. The resulting solutions were filter-sterilized and stored at 4° C. prior to use. ReTain® is listed as 15% AVG-HCl, and the measured weights of this product were adjusted to give the indicated molar amounts of AVG.

### B. Results

Inhibition of Erwinia amylovora by the Naturally-Occurring Vinylglycine, AVG.

The ability of formylaminooxyvinylglycine (FVG) and FVG-producing bacteria to inhibit multiplication of E. amylovora suggested that other vinylglycines and vinylglycine-producing bacteria might exhibit similar activity. Vinylglycine compounds are known to be produced by particular strains of several bacterial species, including both Pseudomonas and non-Pseudomonas species. For example, methoxyvinylglycine (2-amino-4-methoxy-but-3-enoic acid) is produced by the opportunistic human pathogen P. aeruginosa (Scannell et al., J. Antibiotics, 25: 58-63, 1972), and the vinylglycine compound known as rhizobitoxine [2-amino-4-(2-amino-3-hydroxypropyl)-but-3-enoic acid] is synthesized by Bradyrhizobium elkanii and by the broad host-range plant pathogen Burkholderia (formerly Pseudomonas) andropogonis (Yasuta et al., Appl. Eviron. Microbiol. 67: 499-5009, 2001). Aminoethoxyvinylglycine (AVG, 2-amino-4-aminoethoxy-but-3-enoic acid) is synthesized by Streptomyces sp. NRRL 533 (Fernandez et al., Microbiology 148: 1413-1420, 2002). These particular bacterial species appear to be unlikely candidates for use as biocontrol agents in the control of fire blight, but the compounds they produce could be useful for this purpose, and other vinylglycine-producing bacteria could prove to have such utility.

AVG is the vinylglycine that is most readily available commercially, because AVG is the active ingredient in the plant growth regulator ReTain®. ReTain® is currently marketed to the fruit and nut industry as an agent that prevents premature fruit drop by inhibiting the synthesis of the plant hormone ethylene. As disclosed in U.S. patent application Ser. No. 12/567,590, ReTain® shares with FVG the ability to produce germination arrest in the seeds of grassy weeds. Therefore, it appeared likely that this compound would have an effect on E. amylovora similar to that of FVG.

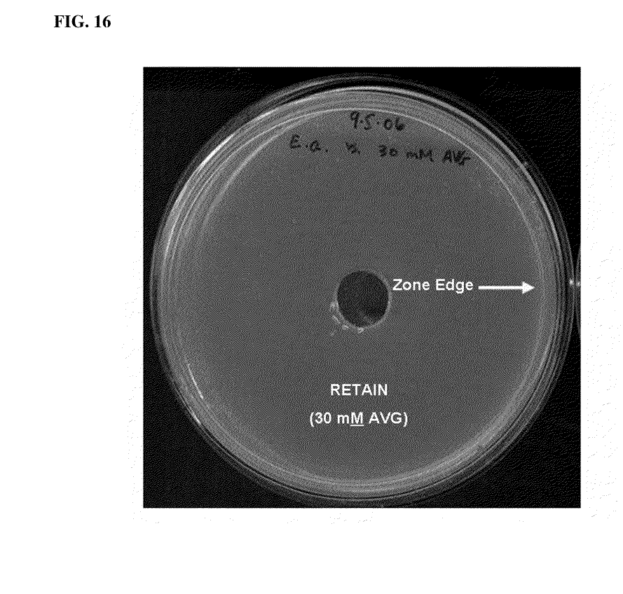

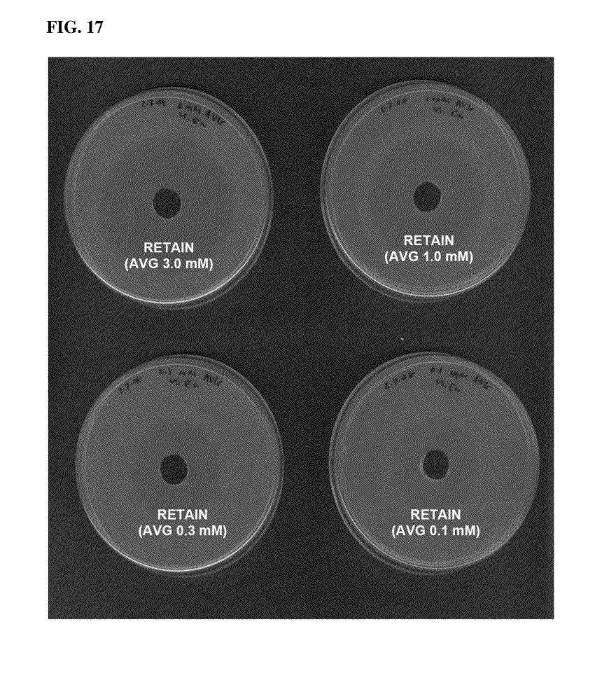

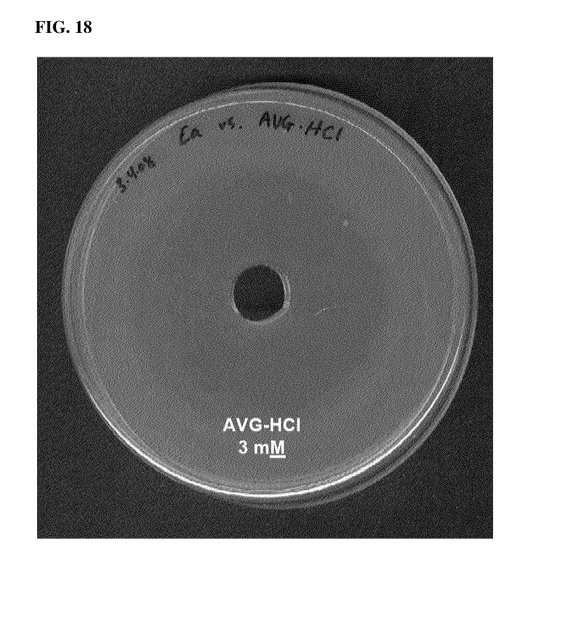

The results of tests of the ability of AVG (supplied as ReTain®) to inhibit the growth of E. amylovora are illustrated in FIGS. 16 and 17. As can be seen from these figures, ReTain had a substantial inhibitory effect on growth of E. amylovora. The inhibitory effect of AVG on E. amylovora has subsequently been confirmed in tests using a commercial sample of pure AVG as shown in FIG. 18.

Inhibitory Effects on Erwinia amylovora of Aminooxyacetic Acid (AOA), a Synthetic Inhibitor of Pyridoxal-Phosphate-Dependent Reactions

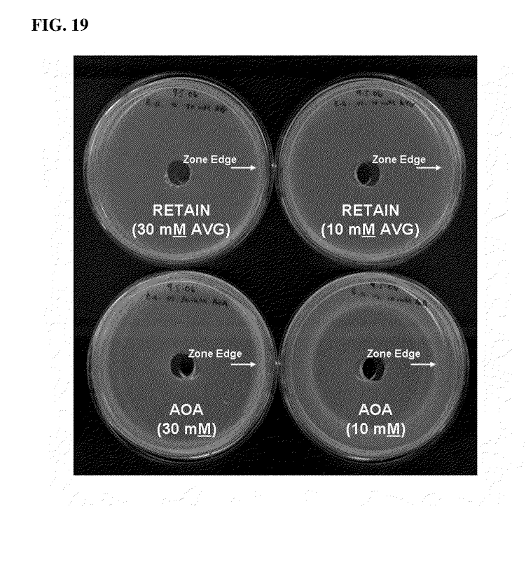

Vinylglycines are known to inhibit pyridoxal-phosphate-dependent enzyme reactions, and it appeared likely that the effects of these compounds on Erwinia amylovora might be mediated by such a mechanism. If this is the case, other types of compounds known to inhibit such reactions would be expected to give a similar effect. To test this hypothesis, the effects of aminooxyacetic acid (AOA), a synthetic inhibitor known to block pyridoxal-phosphate dependent reactions, was tested in the E. amylovora growth inhibition assay. As shown in FIG. 19, AOA was an effective growth inhibitor of E. amylovora. This observation suggests that the ability to inhibit growth of E. amylovora will extend to all compounds that inhibit pyridoxal-phosphate-dependent enzyme reactions by the same mechanism as vinylglycines and AOA.

### Example 3

### Structural and Mechanistic Analogs of FVG, AVG, and AOA as Control Agents of Erwinia amylovora

This example illustrates that structural variants of FVG can be used as control agents of Erwinia amylovora. Likewise, this example describes how mechanistic variants of FVG, AVG, and AOA, which inhibit pyridoxal-phosphate-dependent reactions, can be used as control agents of Erwinia amylovora.

The results with FVG and AVG suggest that the ability to inhibit growth of Erwinia amylovora is a general property of vinylglycines. Thus, many of the vinylglycine structural variants that are illustrated above in section 111D are expected to have the same antibiotic effect of FVG. This activity can be tested using the methods described in Example 1. Each synthesized vinylglycine variant will be filter sterilized and applied to a well that is bored from a lawn of Erwinia amylovora grown on an LB-agar plate. After 48 hours incubation, the ability to control bacterial growth will be determined by comparison of any resultant zone of bacterial inhibition with the inhibition produced using purified FVG or sterilized Pseudomonas fluorescens WH6 culture filtrate. A comparable zone of bacterial inhibition will indicate that the particular vinylglycine structural variant can be used to control the growth of E. amylovora.

Similarly, the results with FVG, AVG, and AOA suggest that other inhibitors of pyridoxal-phosphate-dependent enzyme reactions inhibit E. amylovora growth. This activity can be tested as described in Example 1. An inhibition of E. amylovora growth by a pyridoxal-phosphate-dependant enzyme inhibitor that is comparable to that of FVG will demonstrate that the particular enzyme inhibitor can also be used as a control agent of E. amylovora.

### Example 4

### Efficacy of Vinylglycine-Producing Organisms in Inhibiting the Development of Fire Blight Symptoms in Test Plants

This example describes the use of vinylglycine-producing bacteria as biocontrol agents for inhibiting the development of fire blight in plants. As discussed above, bacteria that possess GAF activity and synthesize FVG are also effective as growth inhibitors of Erwinia amylovora. As E. amylovora is the causal agent of fire blight, it follows that vinylglycine producing bacteria, and in a particular embodiment, FVG-producing bacteria, will be effective at controlling the development of fire blight in plants.

In a particular embodiment, plants that are susceptible to E. amylovora infection, such as apple trees at the tight cluster stage or pear trees at the bud burst stage, are exposed to E. amylovora in a manner and concentration sufficient to induce fire blight disease. Following exposure to the bacteria, the plants are treated with an effective amount of a FVG-producing bacterial strain, such as Pseudomonas fluorescens biotype WH6. Alternatively, the trees are treated with an effective amount of a FVG-producing bacterial strain, such as Pseudomonas fluorescens biotype WH6, prior to exposure to the fire blight pathogen E. amylovora. FVG-producing bacteria can be applied to the plant in any suitable medium or composition known to the art. Additionally, the FVG-producing bacteria may be applied in isolation or together with other known effective biocontrol agents. In this example, the efficacy of P. fluorescens WH6 at controlling the development of fire blight is determined by comparing WH6-treated and untreated plants that have been exposed to E. amylovora, in addition to WH6-treated and untreated plants that have not been exposed to E. amylovora. A reduction or elimination in the characteristic fire blight symptoms (which consist of water soaking of the blossoms and leaf buds followed by rapid wilting and necrosis) in WH6-treated plants that were also exposed to E. amylovora will demonstrate that such FVG-producing organisms are effective at inhibiting or preventing fire blight in plants. Likewise, a similar result obtained by pre- or post-treatment of E. amylovora-exposed plants with bacteria that produce AVG or another vinylglycine molecule will demonstrate that the ability to inhibit fire blight in plants is a general characteristic of vinylglycine-producing bacteria.

### Example 5

### Efficacy of FVG and its Structural Variants in Inhibiting the Development of Fire Blight Symptoms in Test Plants

This example describes the use of vinylglycines to control the development of fire blight in plants. As discussed above, FVG and vinylglycine structural variants such as AVG are growth inhibitors of E. amylovora, the causal agent of fire blight in Rosaceae plants. From this proven ability to control the causal agent of fire blight, it follows that FVG and its structural variants will be effective at inhibiting the development of fire blight in a plant.

Plants that are susceptible to E. amylovora infection, such as apple trees at the tight cluster stage or pear trees at the bud burst stage, are exposed to E. amylovora in a manner and concentration sufficient to induce fire blight disease. Following exposure to the bacteria, the plants are sprayed with an effective amount of FVG or a structural variant such as AVG. Alternatively, an effective amount of FVG or a structural variant such as AVG are sprayed on the trees prior to exposure to the fire blight pathogen E. amylovora. The FVG or structural variant is applied in a purified form or derived from the culture filtrate of bacteria that has GAF activity. It may also be applied as part of a suitable chemical formulation or may be included with other antibiotics that are effective at inhibiting E. amylovora growth. The efficacy of vinylglycine treatment at controlling development of fire blight is determined by comparing treated and untreated plants that have been exposed to E. amylovora, in addition to treated and untreated plants that have not been exposed to E. amylovora. A reduction or elimination in the characteristic fire blight symptoms (which consist of water soaking of the blossoms and leaf buds followed by rapid wilting and necrosis) in E. amylovora-exposed and vinylglycine-treated plants will demonstrate that such treatments are effective at inhibiting or preventing fire blight in plants.

### Example 6

### Efficacy of Pyridoxal-Phosphate-Dependent Enzyme Reaction Inhibitors (Other than Vinylglycines) in Inhibiting the Development of Fire Blight Symptoms in Test Plants

This example describes the use of pyridoxal-phosphate-dependent enzyme reaction inhibitors (other than vinylglycines) to control the development of fire blight in plants. As discussed above, vinylglycines are known to inhibit pyridoxal-phosphate-dependent enzyme reactions. Moreover, as disclosed above, the non-vinylglycine pyridoxal-phosphate-dependent enzyme inhibitor AOA is an effective growth inhibitor of E. amylovora. As E. amylovora is the causal agent of fire blight, it follows that AOA and other non-vinylglycine pyridoxal-phosphate-dependent enzyme inhibitors will be similarly effective at inhibiting the development of fire blight in plants.

Plants that are susceptible to E. amylovora infection, such as apple trees at the tight cluster stage or pear trees at the bud burst stage, are exposed to E. amylovora in a manner and concentration sufficient to induce fire blight disease. Following exposure to the bacteria, the plants are sprayed with an effective amount of AOA or a mechanistic analog. Alternatively, the plants may be sprayed with an effective amount of AOA or a mechanistic analog prior to exposure to the fire blight pathogen E. amylovora. The AOA may be applied as part of a suitable chemical formulation or be included with antibiotics that are effective E. amylovora growth inhibitors. The efficacy of AOA treatment at controlling the development of fire blight is determined by comparing treated and untreated plants that have been exposed to E. amylovora, in addition to treated and untreated plants that have not been exposed to E. amylovora. A reduction or elimination in the characteristic fire blight symptoms (which consist of water soaking of the blossoms and leaf buds followed by rapid wilting and necrosis) in E. amylovora-exposed and AOA-treated plants will demonstrate that such treatments are effective at inhibiting or preventing fire blight in plants.

It will be apparent that the precise details of the methods, uses, and kits described may be varied or modified without departing from the spirit of the described invention. We claim all such modifications and variations that fall within the scope and spirit of the claims below.

We claim:

1. A method of inhibiting growth of E. amylovora on a plant comprising applying to the plant an effective amount of a composition comprising:
a natural or synthetic FVG or a structurally related analog thereof;
a non-vinylglycine inhibitor of pyridoxal-phosphate-dependent enzyme reactions;
a bacterial strain that is producing FVG or a structurally related analog thereof;
prior to fruit set and petal fall from the plant, an effective amount of AVG or a structurally related analog thereof; or
prior to fruit set and petal fall from the plant, a bacterial strain that is producing AVG or a structurally related analog thereof.

2. The method of claim 1, wherein the plant has fire blight.

3. The method of claim 2, wherein the plant is a member of family Rosaceae.

4. The method of claim 3, wherein the plant is an apple tree.

5. The method of claim 3, wherein the plant is a pear tree.

6. The method of claim 1, wherein the bacterial strain is an FVG-producing strain of Pseudomonas fluorescens, an FVG-producing strain of Pseudomonas mucidolens/synxantha, or an FVG producing strain of Enterobacter kobei.

7. The method of claim 6 wherein the bacterial strain is selected from the group consisting of Pseudomonas fluorescens strain isolates WH6 (NRRL# B-30485), AD31 (NRRL# B-30483), AH4 (NRRL#B-30482), E34 (NRRL# B-30481), WH19 (NRRL# B-30484), AH10 (NRRL# B-50232), BT1 (NRRL#B-50230), E24 (NRRL# B-50229), TR33 (NRRL# B-50220), TR44 (NRRL# B-50219), TR46 (NRRL# B-50218), A3422A (NRRL# B-50234), ALW38 (NRRL# B-50231), G2Y (NRRL# B-50228), GTR12 (NRRL# B-50227), GTR24 (NRRL# B-50226), GTR40 (NRRL# B-50225), HB14 (NRRL# B-50224), HB26 (NRRL# B-50223), HB32 (NRRL# B-50222), ST22 (NRRL# B-50221), W36 (NRRL# B-50217) or Pseudomonas mucidolens/synxantha A342 (NRRL# B-50236) or TDH40 (NRRL# B-50235), or Enterobacter kobei A3203 (NRRL# B-50233) and mixtures of two or more thereof.

8. The method of claim 1, wherein the bacteria is applied in an agronomically-compatible composition.

9. The method of claim 1, wherein the FVG, non-vinylglycine inhibitor of pyridoxal-phosphate-dependent enzyme reactions, AVG, or structurally related analog of FVG or AVG is applied in a formulation that also comprises a surfactant, a stabilizer, a buffer, a preservative, an antioxidant, an extender, a solvent, an emulsifier, an invert emulsifier, a spreader, a sticker, a penetrant, a foaming agent, an anti-foaming agent, a thickener, a safener, a compatibility agent, a crop oil concentrate, a viscosity regulator, a binder, a tacker, a drift control agent, a fertilizer, an antibiotic, a fungicide, a nematicide, or a pesticide.

10. The method of claim 1, wherein the FVG, non-vinylglycine inhibitor of pyridoxal-phosphate-dependent enzyme reactions, AVG, or structurally related analog of FVG or AVG is applied in a formulation that is a solution, a soluble powder, an emulsifiable concentrate, a wettable powder, a liquid flowable, a dry flowable, a water-dispersible granule, a granule, or a pellet.

11. The method of claim 1, wherein the FVG, non-vinylglycine inhibitor of pyridoxal-phosphate-dependent enzyme reactions, AVG, or structurally related analog of FVG or AVG is formulated as a granule.

12. The method of claim 11, wherein the granule is at least partially coated with a timed- or temperature-release coating.

13. The method of claim 12, wherein the timed- or temperature-release coating is coated with a water-resistant coating.
